SEQUENCE LISTING (30 sequences)

SEQ ID NO: 1 - DNA, 1327 bp, Vitis vinifera
gggggtgctcagcagagcatcagcttcatagctttagttttcctttctgtcccttgattctctctcactttctcgtgtcccaaacgcgggaaagaaaataaacaaaagttgtacttgtagagtgataagggaaagaaaggaaagagagagatatgggcagagcaccttgttgttctaaggttggtttgcatagaggttcatggactgccagagaagacacattgctgaccaagtatattcaggctcatggtgaaggccactggagatctctccccaagaaagctggtcttttgagatgtgggaagagttgcaggctgagatggatgaattatctaaggccggatatcaaaagaggaaacatcacgccggacgaagacgacctaattatcagactgcattcacttttgggaaatcggtggtctctcatcgcaggtaggcttccaggtcgaaccgataatgagatcaagaactactggaacacccatctgagtaagaagctaagaagtcaaggaacagaccccaatacccacaagaaaatgacagagcctcctgagcccaagaggaggaagaacacaagaacaagaaccaacaatggcggaggcagcaagagggtcaagatcagcaaggacgaagaaaattccaatcacaaagtccatctccccaaaccggttagagtcacctccttaatctccatgtcgaggaacaacagctttgaatctaatacagtcagcgggggctcgggctcgagttcaggaggaaatggggaatcgcttccatggcctagtttcagagacatccgggacgacaaggttattggggttgacggggttgacttcttcatcggagatgatcaaggtcaagacctggtcgccagctccgatccagaatcacaatcccacatgccaccaactgacaactctctggacaaactgtatgaagagtatctgcaactgcttgagcgagaagacactcaagttcagcttgactccttcgccgaatcactactcatttaattcaaatttcaaaaggagttgcagagagaccaaaagatcaactggttatgcttgcttgcttgctttgaatcttagcttccatgcacgtgctcaccttcttcaaaccctagcttggttatgaacttcattgatctattaattaattacttagcttatatatcttattagtttagtttttaatcttataatagcgatacgtgcggtgtgcgatgtacatttgtgttaataatatggtttgtaaggtgggtattaacaaacaaatgagccaaccgaagcgtcccgattattcgaccaattgataaatttgattaatt

SEQ ID NO: 2 - protein, 286 aa, Vitis vinifera
MGRAPCCSKVGLHRGSWTAREDTLLTKYIQAHGEGHWRSLPKKAGLLRCGKSCRLRWMNYLRPDIKRGNITPDEDDLIIRLHSLLGNRWSLIAGRLPGRTDNEIKNYWNTHLSKKLRSQGTDPNTHKKMTEPPEPKRRKNTRTRTNNGGGSKRVKISKDEENSNHKVHLPKPVRVTSLISMSRNNSFESNTVSGGSGSSSGGNGESLPWPSFRDIRDDKVIGVDGVDFFIGDDQGQDLVASSDPESQSHMPPTDNSLDKLYEEYLQLLEREDTQVQLDSFAESLLI

SEQ ID NO: 3 - DNA, 31 bp, Artificial Sequence
  Forward Primer Directed to Vitis vinifera
tgaggtaccgagagagatatgggcagagcac

SEQ ID NO: 4 - DNA, 31 bp, Artificial Sequence
  Reverse Primer Directed to Vitis vinifera
tgaggatcctgatcttttggtctctctgcaa

SEQ ID NO: 5 - DNA, 35 bp, Artificial Sequence
  Forward Primer, with BamHI restriction site, Directed to Vitis vinifera
ataggatcctggaattatggaagacaaatagtcaa

SEQ ID NO: 6 - DNA, 30 bp, Artificial Sequence
  Reverse Primer, with XhoI restriction site, directed to Vitis vinifera
ttactcgaggatatggctgcagagaaacga

SEQ ID NO: 7 - DNA, 36 bp, Artificial Sequence
  Forward Primer, with BamHI restriction site, directed to Vitis vinifera
cgaggatcccattcatagtcaaattacaaaaatcaa

SEQ ID NO: 8 - DNA, 31 bp, Artificial Sequence
  Reverse Primer, with XhoI restriction site, directed to Vitis vinifera
atactcgagatatgccctcacttccaaattc

SEQ ID NO: 9 - DNA, 33 bp, Artificial Sequence
  Forward Primer, with BamHI restriction site, directed to Vitis vinifera
cgaggatcccaaaaagagttggaaatacaacga

SEQ ID NO: 10 - DNA, 36 bp, Artificial Sequence
  Reverse Primer, with XhoI restriction site, directed to Vitis vinifera
atactcgagtgagtataggatagtgaaggtggctat

SEQ ID NO: 11 - DNA, 32 bp, Artificial Sequence
  Forward Primer, with BamHI restriction site, directed to Vitis vinifera
cgaggatcctcggaataatttcatagggcttt

SEQ ID NO: 12 - DNA, 36 bp, Artificial Sequence
  Reverse Primer, with XhoI restriction site, directed to Vitis vinifera
atactcgagtctgatgatgcttcttctctactactc

SEQ ID NO: 13 - DNA, 31 bp, Artificial Sequence
  Forward Primer, with BamHI restriction site, directed to Vitis vinifera
acgggatcctcatgcgtccacctattatcaa

SEQ ID NO: 14 - DNA, 29 bp, Artificial Sequence
  Reverse Primer, with XhoI restriction site, directed to Vitis vinifera
gtactcgagggttggaatgggggatgtta

SEQ ID NO: 15 - DNA, 29 bp, Artificial Sequence
  Forward Primer, with BamHI restriction site, directed to Vitis vinifera
cgaggatccctgggaagacaatcgcttta

SEQ ID NO: 16 - DNA, 37 bp, Artificial Sequence
  Reverse Primer, with XhoI restriction site, directed to Vitis vinifera
atctcgagttgaaattacagagatagagatttagttg

SEQ ID NO: 17 - DNA, 33 bp, Artificial Sequence
  Forward Primer, with BamHI restriction site, directed to Vitis vinifera
cgaggatccgtttgcttccatcccaatctcact

SEQ ID NO: 18 - DNA, 35 bp, Artificial Sequence
  Reverse Primer, with XhoI restriction site, directed to Vitis vinifera
tgtctcgagaaatatcactgatctacttgttttcc

SEQ ID NO: 19 - DNA, 33 bp, Artificial Sequence
  Forward Primer, with BamHI restriction site, directed to Vitis vinifera
aggtcgacatgggaaagagagcaactactagtg

SEQ ID NO: 20 - DNA, 30 bp, Artificial Sequence
  Reverse Primer, with XhoI restriction site, directed to Vitis vinifera
tactcgagtcaacaagtgaagtctcggagc

SEQ ID NO: 21 - DNA, 33 bp, Artificial Sequence
  Forward Primer, with BamHI restriction site, directed to Vitis vinifera
cgcctcgagctcgatggagagcttaggagttag

SEQ ID NO: 22 - DNA, 33 bp, Artificial Sequence
  Reverse Primer, with XhoI restriction site, directed to Vitis vinifera
cgctctagataaatcagatcaaatgatttactt

SEQ ID NO: 23 - DNA, 22 bp, Artificial Sequence
  Forward Primer directed to Vitis vinifera
gtggtattattgagccatcctt

SEQ ID NO: 24 - DNA, 22 bp, Artificial Sequence
  Reverse directed to Vitis vinifera
aacctccaatccagtcatctac

SEQ ID NO: 25 - DNA, 22 bp, artificial sequence
  Forward Primer directed to Vitis vinifera
agatcaactggttatgcttgct

SEQ ID NO: 26 - DNA, 22 bp, artificial sequence
  Reverse Primer directed to Vitis vinifera
aacacaaatgtacatcgcacac

SEQ ID NO: 27 - DNA, 22 bp, artificial sequence
  Forward Primer directed to Arabidopsis
caactgacaactctctggacaa

SEQ ID NO: 28 - DNA, 22 bp, artificial sequence
  Reverse Primer directed to Arabidopsis
gatcttttggtctctctgcaac

SEQ ID NO: 29 - DNA, 26 bp, artificial sequence
  Forward Primer directed to Arabidopsis
attcagatgcccagaagtcttgttcc

SEQ ID NO: 30 - DNA, 26 bp, artificial sequence
  Reverse Primer directed to Arabidopsis
accaccgatccagacactgtacttcc

(19) United States
(12) Patent Application Publication
(10) Pub. No.: US 2012/0208198 A1
(43) Pub. Date: Aug. 16, 2012

(54) NOVEL GENE ENCODING MYB TRANSCRIPTION FACTOR INVOLVED IN PROANTHOCYANIDIN SYNTHESIS

(75) Inventors: Jochen Bogs, Heidelberg (DE); Amanda Ruth Walker, Upper Sturt (AU); Simon Piers Robinson, Hawthorn (AU)

(73) Assignee: COMMONWEALTH SCIENTIFIC AND INDUSTRIAL RESEARCH ORGANIZATION

(21) Appl. No.: 13/346,478

(22) Filed: Jan. 9, 2012

Related U.S. Application Data
(62) Division of application No. 12/008,496, filed on Jan. 11, 2008, now abandoned.
(60) Provisional application No. 60/880,177, filed on Jan. 11, 2007.

(51) Int. Cl. C12Q 1/68

(52) U.S. Cl. 435/6.12; 435/6.1

(57) ABSTRACT

An isolated or recombinant MYB polypeptide having activity as a transcription factor in the synthesis of proanthocyanidins in plants, and nucleic acid molecule encoding same, wherein the polypeptide activates in the plants (a) promoters of the leucoanthocyanidin (LAR) and anthocyanidid reductase (ANR) genes, and (b) promoters of at least two of the genes of the general flavonoid pathway. Use of the polypeptide and nucleic acid molecule in regulating the biosynthesis and accumulation of proanthocyanidins in plants, such as in modifying pasture quality of legumes, is also disclosed.

This application claims benefit of U.S. Provisional Application No. 60/880,177, filed Jan. 11, 2007, the contents of which are hereby incorporated by reference.

### FIELD OF THE INVENTION

The present invention relates generally to isolated proteins or polypeptides which are involved in proanthocyanidin (PA) synthesis in plants, and to nucleic acid molecules encoding same and their use in regulating the biosynthesis and accumulation of proanthocyanidins in plants. The isolated proteins or polypeptides and nucleic acid molecules of the present invention are useful for modifying the pasture quality of legumes, and, in particular, for producing bloat-safe forage crops, or crops having enhanced nutritional value, enhanced disease resistance or pest resistance. In addition, these isolated proteins or polypeptides and nucleic acid molecules are useful in enhancing dietary PAs in fruits and plant products such as wine, fruit juices and teas.

### General

Those skilled in the art will be aware that the invention described herein is subject to variations and modifications other than those specifically described. It is to be understood that the invention described herein includes all such variations and modifications. The invention also includes all such steps, features, compositions and compounds referred to or indicated in this specification, individually or collectively, and any and all combinations of any two or more of said steps or features.

Throughout this specification, unless the context requires otherwise the word “comprise”, and variations such as “comprises” and “comprising”, will be understood to imply the inclusion of a stated integer or step or group of integers or steps but not the exclusion of any other integer or step or group of integers or steps. The present invention is not to be limited in scope by the specific embodiments described herein, which are intended for the purposes of exemplification only. Functionally-equivalent products, compositions and methods are clearly within the scope of the invention, as described herein.

Bibliographic details of the publications referred to by author in this specification are collected at the end of the description. Reference herein to prior art, including any one or more prior art documents, is not to be taken as an acknowledgment, or suggestion, that said prior art is common general knowledge in Australia or forms a part of the common general knowledge in Australia.

As used herein, the term “derived from” shall be taken to indicate that a particular integer or group of integers has originated from the species specified, but has not necessarily been obtained directly from the specified source.

This specification contains nucleotide sequence information prepared using the program PatentIn Version 3.1, presented herein after the claims. Each nucleotide sequence is identified in the sequence listing by the numeric indicator <210> followed by the sequence identifier (e.g. <210>1, <210>2, etc). The length, type of sequence (DNA, protein (PRT), etc) and source organism for each nucleotide sequence are indicated by information provided in the numeric indicator fields <211>, <212> and <213>, respectively. Nucleotide sequences referred to in the specification are defined by the term “SEQ ID NO:”, followed by the sequence identifier (e.g. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymidine, Y represents a pyrimidine residue, R represents a purine residue, M represents Adenine or Cytosine, K represents Guanine or Thymidine, S represents Guanine or Cytosine, W represents Adenine or Thymidine, H represents a nucleotide other than Guanine, B represents a nucleotide other than Adenine, V represents a nucleotide other than Thymidine, D represents a nucleotide other than Cytosine and N represents any nucleotide residue.

### BACKGROUND TO THE INVENTION

Proanthocyanidins (PAs), also known as condensed tannins, are polyphenolic secondary metabolites synthesized via the flavonoid biosynthetic pathway. They are present in many plants and act in defence against plant diseases and in seed dormancy (Peters and Constabel 2002; Debeaujon et al., 2000). Dietary PAs are present in many fruits and plant products like wine, fruit juices and teas and contribute to their taste and health benefits. PAs act as potential dietary antioxidants with beneficial effects for human health including protection against free radical-mediated injury and cardiovascular disease (Middleton et al., 2000; Bagchi et al., 2000; Cos et al., 2004). There is also considerable interest in the PAs found in grape skins because of their importance for the flavor and astringency of red and white wine (Glories, 1988). Furthermore, the increase of PAs in important forage crops like alfalfa could protect ruminants against pasture bloat, reduce greenhouse gas and increase plant disease resistance (Dixon et al., 1996; McMahon et al., 2000). For these reasons, there is a growing interest in metabolic engineering strategies aimed at developing agronomically important food crops and fruits with optimized levels and composition of flavonoids.

The biosynthesis of PAs, anthocyanins and flavonols share common steps in the flavonoid pathway and the genetics and biochemistry of this pathway (FIG. 1) have been characterized in several plant species including Arabidopsis thaliana and Vitis vinifera (Shirley et al., 1992; Holton and Cornish, 1995; Boss et al., 1996; Winkel-Shirley, 2001).

In Arabidopsis, the biosynthetic pathway leading to PA accumulation has been characterized by using the transparent testa (tt) and tannin-deficient seed (tds) mutants which fail to accumulate PAs in their seed coat (Shirley et al., 1995; Abrahams et al., 2002). The identified tt and tds loci correspond to enzymes of the general flavonoid pathway and to enzymes, transporters and regulators specifically involved in PA accumulation. The structural genes include ANR (also called BANYULS) that catalyzes the synthesis of flavan-3-ols such as (−)-epicatechin (Xie et al., 2003), TT19, TT12 and AHA10 which are involved in transport processes of PAs (Debeaujon et al., 2001; Kitamura et al., 2004; Baxter et al., 2005) and TT10 encoding a laccase-type polyphenol oxidase involved in polymerization of flavonoids (Pourcel et al., 2005).

Most of the regulation of flavonoid synthesis occurs via coordinated transcriptional control of the structural genes by the interaction of DNA-binding MYB transcription factors and MYC-like basic helix-loop-helix (bHLH) proteins (Mol at al., 1998; Nesi at al., 2000 and 2001, Winkel-Shirley at al., 2001). This has been shown for several regulators of anthocyanin synthesis isolated from maize, Arabidopsis, Antirrhinum and petunia (Mol et al., 1998). The genes TT8, TT2 and TTG1 were found to encode a basic helix-loop-helix (bHLH), an R2R3MYB-type and a WD40-repeat protein, respectively. These transcription factors are necessary for PA accumulation in the seed coat of Arabidopsis and regulate the expression of several flavonoid structural genes including ANR and TT12 (Nesi et al., 2000 and 2001, Walker et al., 1999; Baudry et al., 2004). Although these structural genes were also induced in roots after ectopic expression of TT2 and TT8, the tissue failed to accumulate PAs suggesting additional factors are required for ectopic PA accumulation in Arabidopsis (Nesi et al., 2001). The transcription factors TT16, TT1 and TTG2 have been shown to influence expression of the PA specific genes and the organ and cell development important for PA deposition (Nesi et al., 2002; Johnson et al., 2002; Sargasser et al., 2002). Recently, the grapevine transcription factor VvMYB5a was shown to affect the metabolism of anthocyanins, flavonols, lignins and PAs in tobacco, suggesting it controls different branches of the phenylpropanoid pathway in grapevine (Deluc et al., 2006).

International Patent Publication WO 2006/010096 discloses the expression of DNA encoding TT2 in a transgenic plant, particularly a forage crop such as alfalfa, whereby the plant exhibits increased condensed tannin (CT) biosynthesis relative to another plant that differs from the transgenic plant only in that the DNA encoding TT2 is absent. This publication also discloses further embodiments on which other CT biosynthesis genes such as a coding sequence for a BAN polypeptide and/or a coding sequence for a chalcone isomerise polypeptide are also expressed in the transgenic plant.

However, the specific regulation of PA synthesis in plant species other than Arabidopsis is not well characterized and until now no functional homologue of TT2 has been identified. Whereas PA synthesis in Arabidopsis is exclusively epicatechin-based and limited to the seed coat, many other plants produce both epicatechin and catechin-based PAs of various amounts and compositions and in a range of different tissues (Dixon et al., 2005). In grapevine, the first committed steps in PA biosynthesis are catalysed by leucoanthocyanidin reductase (LAR) and anthocyanidin reductase (ANR) by converting anthocyanidins to flavan-3-ols such as catechin and epicatechin, respectively. Grapevine synthesizes PAs in various compositions in the seeds and skin of the fruit where their accumulation occurs during early grape berry development. The tissue and temporal-specific expression of ANR and LAR correlates with PA accumulation in grapes (Bogs et al., 2005) suggesting a transcriptional regulator is controlling PA synthesis in grapevine.

### SUMMARY OF THE INVENTION

In work leading up to the present invention, the inventors have isolated and characterized the grapevine gene VvMYBPA1 encoding a MYB transcription factor which is expressed when PAs accumulate during grape berry development. VvMYBPA1 is able to activate the promoters of general flavonoid pathway genes and the branch point genes VvANR and VvLAR1 which were shown to be involved in PA synthesis of grapevine. Further, the inventors have shown that VvMYBPA1 is able to complement the PA deficient phenotype of the Arabidopsis tt2 mutant and to induce ectopic PA accumulation in Arabidopsis.

Accordingly, in one aspect the present invention provides an isolated or recombinant MYB polypeptide having activity as a transcription factor in the synthesis of proanthocyanidins in plants, wherein said polypeptide activates in said plants (a) promoters of the leucoanthocyanidin (LAR) and anthocyanidin reductase (ANR) genes, and (b) promoters of at least two of the genes of the general flavonoid pathway.

In one embodiment, said polypeptide comprises an amino acid sequence substantially corresponding to the VvMYBPA1 protein sequence set forth in SEQ ID NO: 2 or an orthologue or homologue thereof, or an amino acid sequence having at least 40% identity overall thereto, or an amino acid sequence having at least 40% identity to amino acids 116-286 of said VvMYBPA1 protein sequence; or a fragment comprising at least about 10 contiguous amino acids derived from said polypeptide.

Preferably, the isolated or recombinant polypeptide is the VvMYBPA1 protein described in detail herein, or a biologically active fragment thereof.

The present invention also provides an isolated nucleic acid molecule comprising (i) a nucleotide sequence that encodes a MYB polypeptide having activity as a transcription factor in the synthesis of proanthocyanidins in plants, wherein said polypeptide activates in said plants (a) promoters of the leucoanthocyanidin (LAR) and anthocyanidin reductase (ANR) genes, and (b) promoters of at least two of the genes of the general flavonoid pathway; or (ii) a nucleotide sequence that encodes a fragment comprising at least about 10 contiguous amino acids derived from said polypeptide; or (iii) a nucleotide sequence that is complementary to (i) or (ii).

In one embodiment, said nucleotide sequence (i) is a sequence that encodes a polypeptide which comprises an amino acid sequence substantially corresponding to the VvMYBPA1 protein sequence set forth in SEQ ID NO: 2 or an orthologue or homologue thereof, or an amino acid sequence having at least 40% identity overall thereto, or an amino acid sequence having at least 40% identity to amino acids 116-286 of said VvNYBPA1 protein sequence.

The isolated nucleic acid molecule may comprise DNA and/or RNA.

The present invention also provides an isolated nucleic acid molecule comprising a nucleotide sequence selected from the group consisting of: (i) a nucleotide sequence having at least about 40% identity overall to the VvMYBPA1 nucleotide sequence set forth in SEQ ID NO: 1 or a protein coding region thereof; (ii) a nucleotide sequence that is complementary to (i); or (iii) a nucleotide sequence that hybridises to at least about 20 contiguous nucleotides of (i) or (ii) under at least low stringency conditions, preferably under moderate stringency conditions and more preferably under high stringency conditions.

Preferably, the isolated nucleic acid molecule comprises the VvMYBPA1 nucleotide sequence described in detail herein, or a fragment thereof, or a sequence complementary to said nucleotide sequence or fragment.

This invention also extends to any synthetic or chimeric gene constructs that comprise the isolated nucleic acid molecule of the present invention, such as, for example, any expression gene constructs produced for expressing said nucleic acid molecule in a bacterial, insect, yeast, plant, fungal, or animal cell. Accordingly, a further aspect of the present invention is directed to a gene construct comprising an isolated nucleic acid molecule as described above. The gene construct preferably comprises the isolated nucleic acid molecule operably linked to a heterologous promoter which is capable of expression in a plant cell, optionally a tissue specific promoter or a promoter that is expressed preferentially in epidermal cells.

A further aspect of the invention contemplates a cell such as a plant cell comprising a non-endogenous nucleic acid molecule or gene construct as described above, preferably wherein said nucleic acid molecule is present in said cell in an expressible format.

A further aspect of the invention contemplates a transformed plant comprising a non-endogenous nucleic acid molecule as described above introduced into its genome, in an expressible format. Preferably, the transformed plant of the invention further expresses a non-endogenous polypeptide encoded by the nucleic acid molecule in at least some cells or tissues, including ectopic expression in cells or tissues in which the polypeptide is not usually expressed. This aspect of the invention clearly extends to any plant parts, or progeny plants comprising the nucleic acid molecule, that are derived from the primary transformed plant.

A still further aspect of the invention contemplates a method of enhancing the expression of a MYB polypeptide in a plant or plant tissues comprising introducing to the genome of said plant a non-endogenous nucleic acid molecule or a gene construct as described above, in an plant-expressible format.

A still further aspect of the invention contemplates a method of reducing the expression of a MYB polypeptide in a plant or plant tissues comprising introducing to the genome of said plant a molecule selected from the group consisting of: an antisense molecule, a PTGS molecule, and a co-suppression molecule, wherein said molecule comprises at least about 20 contiguous nucleotides of a nucleic acid molecule or complementary to a nucleic acid molecule as described above, in an plant-expressible format.

A still further aspect of the invention contemplates a method of reducing the expression of a MYB polypeptide in a plant or plant tissues comprising introducing to the genome of said plant a ribozyme molecule, wherein said molecule comprises at least two hybridising regions each of at least 5 contiguous nucleotides complementary to a nucleic acid molecule as described above, separated by a catalytic domain capable of cleaving an RNA encoding said polypeptide, in an plant-expressible format.

The present invention further extends to the use of the transformed plants and methods described herein for the purposes as described herein. In particular, the present invention extends to the use of the transformed plants as animal food in fodder in order to improve bloat safety, increase efficiency of protein utilisation and/or improve disease- or pest-resistance in animals. The present invention also extends to the production of food plants and fruits having improved anti-oxidant and free radical scavenging properties, as well as longer shelf life.

### BRIEF DESCRIPTION OF THE DRAWINGS

FIG. 1. Scheme of the flavonoid pathway leading to synthesis of anthocyanins, flavonols and proanthocyanidins. The enzymes involved in the pathway are shown as follows: CHS, chalcone synthase; CHI, chalcone isomerase; F3′H, flavonoid-3′-hydroxylase; F3′5′H, flavonoid-3′,5′-hydroxylase; F3H, flavanone-3β-hydroxylase; DFR, dihydroflavonol-4-reductase; LDOX, leucoanthocyanidin dioxygenase; FLS, flavonol synthase; LAR, leucoanthocyanidin reductase and ANR, anthocyanidin reductase; UFGT, UDP-glucose:flavonoid-3-O-glucosyltransferase. The flavonoid-3′-hydroxylase (F3′H) and flavonoid-3′,5′-hydroxylase (F3′5′H) enzymes, which hydroxylate flavanones and dihydroflavonols, were omitted to clarify the scheme of the flavonoid pathway (see Bogs et al., 2006).

FIGS. 2A-2B: (A) Alignment of the deduced amino acid sequences of the MYB-type transcriptional regulators ZmC1 (maize), AtTT2, AtPAP1, AtMYB12 (Arabidopsis) and the grapevine regulators VvMYBPA1, VvMYBA2 and VvMYB5a. The R2 and R3 repeats of the MYB domain are indicated below the alignment. Identical amino acids are indicated in black, similar amino acids in gray. Sequences were aligned with the ClustalW program and displayed using the GeneDoc program (Version 2.6.002). (B) Phylogenetic tree showing selected plant MYB transcription factors from GenBank or EMBL database. Functions of some of the proteins are given in boldface. The ClustalW multiple sequence alignment was formed using the R2R3 domain of the MYB proteins and the default parameters of the MEGA package (Kumar et al., 2004). The tree was constructed from the ClustalW alignment using the Neighbor-Joining method by the MEGA program. The scale bar represents 0.1 substitutions per site and the numbers next to the nodes are bootstrap values from 1000 replicate. The Genbank accession numbers of the MYB proteins are as follows: VvMYBPA1 (AM259485), AtGL1 (P27900), ZmP (P27898), ZmC1 (AAA33482), VvMYBA1 (BAD18977), VvMYBA2 (BAD18978), AtPAP1 (AAG42001), PhAN2 (AAF66727), LeANT1 (AAQ55181), OsMYB4 (T02988), AtMYB5 (U26935), PhMYB1 (Z13996), AmMixta (CAA55725), AtMYB12 (CAB09172), AtMYB111 (AAK97396), PmMBF1 (AAA82943), AtTT2 (Q9FJA2), PH4 (AAY51377), AtPAP1 (AAG42001), AtPAP2 (AAG42002), AtWER (CAC01874), VvMYB5a (AAS68190), VvMYB5b (Q58QD0).

FIG. 3. Transcript levels of VvMYBPA1 during grape flower and berry development. Gene expression was determined by Real Time PCR and is shown relative to expression of VvUbiquitin1 in each sample. Grey bars represent gene expression levels in buds and flowers, open bars in skins and black bars in seeds. All data is presented as mean of three replicates with error bars indicating±standard errors.

FIGS. 4A-4G: VvMYBPA1 activates promoters of flavonoid pathway genes involved in PA synthesis. The MYB transcription factors and promoters used for transfection of grape cell cultures are indicated and listed herein: AtANR (A), VvANR (B), VvLAR1 (C), VvF3′5′H1 (D), VvLDOX (E), VvCHI (F), and VvUFGT (G). Control indicates the activity of the respective promoter transfected without a MYB factor. Each transfection (except E, MybPA1 w/o bHLH) contained the 35S::EGL3 construct encoding the bHLH protein EGL3 (GB accession: NM20235) from Arabidopsis and as internal control the Renilla luciferase plasmid pRluc (Horstmann et al., 2004). The normalized luciferase activity was calculated as the ratio between the firefly and the Renilla luciferase activity. Each column represents the mean value of three independent experiments with error bars indicating±standard errors.

FIGS. 5A-5B: VvMYBPA1 expression in Arabidopsis tt2 mutant induced PA accumulation in developing siliques. (A) Detection of VvMYBPA1 transcript in tt2 mutant, Col-0 wild-type, and tt2 35S::MYBPA1 lines 10F, 10-2, 10A, 17D, 17B and 17K by RT-PCR. Expression of the Arabidopsis Actin2 gene was used as a positive control. (B) Quantification of PAs (total epicatechins) after acid-catalysed hydrolysis of PA polymers in siliques of Arabidopsis tt2, Col-0 wild-type and tt2 35S::MYBPA1 lines 10F, 10-2, 10A, 17D, 17B and 17K by HPLC. Technical replicates could not be performed due to the limited amount of silique tissue.

FIGS. 6A-6D: Functional analysis of VvMYBPA1 gene expression in tobacco. (A) Proanthocyanidin (PA) accumulation after DMACA staining. (B) Quantification of PA levels in petals using DMACA reagent. (C) Anthocyanin and flavonol content in petals determined by reverse-phase HPLC. (B) Flavonol composition in petals determined by reverse-phase HPLC.

### DETAILED DESCRIPTION OF THE INVENTION

Proanthocyanidins (PAs or condensed tannins) can protect plants against herbivores, contribute to the taste of many fruits and act as potential dietary antioxidants with beneficial effects for human health. During grape berry development the genes encoding enzymes specifically involved in synthesis of PAs are only expressed when PAs accumulate. The present inventors have isolated the gene VvMYBPA1 encoding a MYB transcription factor from grapevine (Vitis vinifera L. cv Shiraz) with a gene expression pattern correlating with PA accumulation during early fruit development and in seeds. In a transient assay, VvMYBPA1 activated the promoters of the PA specific genes VvLAR1 and VvANR encoding leucoanthocyanidin reductase (LAR) and anthocyanidin reductase (ANR), as well as promoters of the general flavonoid pathway genes. The promoter of VvUFGT encoding the anthocyanin specific enzyme UDP glucose:flavonoid-3-O-glucosyltransferase was not activated by VvMYBPA1 showing its specificity to regulation of PA biosynthesis. The MYB transcription factor TT2 (TRANSPARENT TESTA 2) from Arabidopsis was shown to regulate PA synthesis in the seed coat of Arabidopsis. By complementing the PA-deficient seed phenotype of the tt2 mutant with VvMYBPA1, the function of VvMYBPA1 was confirmed as a transcriptional regulator of PA synthesis.

In one aspect the present invention provides an isolated or recombinant MYB polypeptide having activity as a transcription factor in the synthesis of proanthocyanidins in plants, wherein said polypeptide activates in said plants (a) promoters of the leucoanthocyanidin (LAR) and anthocyanidin reductase (ANR) genes, and (b) promoters of at least two of the genes of the general flavonoid pathway.

These MYB polypeptides are referred to herein, for convenience, as “MYBPA1 proteins”. The MYBPA1 proteins of the present invention have been shown to induce promoters of the whole flavonoid pathway, including both “early” biosynthetic genes (EBCs) and “late” biosynthetic genes (LBGs), as well as inducing the promoters of the branch point LAR and AWR genes. In one embodiment, said polypeptide comprises an amino acid sequence substantially corresponding to the VvMYBPA1 protein sequence set forth in SEQ ID NO: 2 or an orthologue or homologue thereof, or an amino acid sequence having at least 40% identity overall thereto or an amino acid sequence having at least 40% identity to amino acids 116-286 of said VvMYBPA1 protein sequence; or a fragment comprising at least about 10 contiguous amino acids derived from said polypeptide.

Preferably, the isolated or recombinant polypeptide is the VvMYBPA1 protein described in detail herein, or a biologically active fragment hereof.

As used herein, the term “fragment” is used to include a biologically active fragment, that is a fragment of a protein or polypeptide having the biological activity of the protein or polypeptide.

Fragments of the isolated MYBPA1 protein of the present invention are useful for the purposes of producing antibodies against one or more B-cell or T-cell epitopes of the protein, which antibodies may be used, for example, to identify cDNA clones encoding homologues of the exemplified cDNA clones provided herein, or in immunohistochemical staining to determine the site of expression of the MYBPA1 protein. Those skilled in the art will appreciate that longer fragments than those consisting of only 10 amino acids in length may have improved utility than shorter fragments. Preferably, a fragment of a MYBPA1 protein of the invention will comprise at least about 20 contiguous amino acid residues, and more preferably at least about 50 contiguous amino acid residues derived from the native protein. Fragments derived from the internal region, the N-terminal region, or the C-terminal region of the native protein are encompassed by the present invention.

Fragments and isolated MYBPA1 proteins contemplated herein include modified peptides in which ligands are attached to one or more of the amino acid residues contained therein, such as a hapten; a carbohydrate; an amino acid, such as, for example, lysine; a peptide or polypeptide, such as, for example, keyhole limpet haemocyanin (KLH), ovalbumin, or phytohaemagglutinin (PHA); or a reporter molecule, such as, for example, a radionuclide, fluorescent compound, or antibody molecule. Glycosylated, fluorescent, acylated or alkylated forms of the subject peptides are particularly contemplated by the present invention. Additionally, homopolymers or heteropolymers comprising two or more copies of the subject MYBPA1 protein are contemplated herein. Procedures for derivatizing peptides are well-known in the art.

Notwithstanding that the present inventors have exemplified the MYBPA1 proteins of the invention from Vitis, the invention clearly extends to isolated MYBPA1 proteins from other plant species, and, in the case of isolated proteins prepared by recombinant means, from any cellular source that supports the production of a recombinant MYBPA1 protein. Accordingly, the present invention clearly encompasses orthologues and homologues of the MYBPA1 proteins and fragments described herein.

In the present context, “homologues” of the MYBPA1 protein of the present invention refer to those proteins having a similar sequence to the MYBPA1 protein, while “orthologues” of the MYBPA1 protein are functionally equivalent homologues, that is homologues which have a similar activity to the MYBPA1 protein, notwithstanding any amino acid substitutions, additions or deletions thereto. An orthologue or homologue of the MYBPA1 proteins exemplified herein may be isolated or derived from the same or another plant species.

For example, the amino acids of a MYBPA1 protein may be replaced by other amino acids having similar properties, for example hydrophobicity, hydrophilicity, hydrophobic moment, charge or antigenicity, and so on. Substitutions encompass amino acid alterations in which an amino acid is replaced with a different naturally-occurring or a non-conventional amino acid residue.

Conservative amino acid substitutions are particularly contemplated herein for the production of orthologues or homologues of the MYBPA1 protein, such as, for example Gly [symbol] Ala; Ser [symbol] Thr; Met [symbol] Val [symbol] Ile [symbol] Lwu; Asp [symbol] Glu; Lys [symbol] Arg; Asn [symbol] Gln; or Phe [symbol] Trp [symbol] Tyr. Such conservative substitutions will not generally inactivate the activity of the MYBPA1 protein.

The non-conservative substitution of one or more amino acid residues in the native MYBPA1 protein for any other naturally-occurring amino acid, or for a non-naturally occurring amino acid analogue, is also contemplated herein. Such substitutions generally involve modifications to charge, in particular charge reversals, or changes to the hydrophobicity of the MYBPA1 protein, and, more preferably, will modify the activity of the protein.

Amino acid substitutions are typically of single residues, but may be of multiple residues, either clustered or dispersed.

Orthologues and homologues of the isolated MYBPA1 proteins, wherein amino acid resides are deleted, or alternatively, additional amino acid residues are inserted are also contemplated herein. Amino acid deletions will usually be of the order of about 1-10 amino acid residues, and may occur throughout the length of the polypeptide. Insertions may be of any length, and may be made to the N-terminus, the C-terminus or be internal. Generally, insertions within the amino acid sequence will be smaller than amino- or carboxyl-terminal fusions and of the order of 1-4 amino acid residues.

The MYBPA1 protein of the present invention may comprise an amino acid sequence having at least about 40% identity overall to the VvMYBPA1 protein sequence described herein, or an amino acid sequence having at least 40% identity to amino acids 116-286 of said VvMYBPA1 protein sequence.

Preferably, the percentage identity to an amino acid sequence presented herein is at least about 50%, more preferably at least about 60%, even more preferably at least about 70%, even more preferably at least about 80%, even more preferably at least about 90%, and even more preferably at least about 95% or 99%.

Those skilled in the art will be aware that the particular percentage identity between two or more amino acid sequences in a pairwise or multiple alignment may vary depending on the occurrence, and length, of any gaps in the alignment. Preferably, for the purposes of defining the percentage identity to the amino acid sequences listed herein, reference to a percentage identity between two or more amino acid sequences shall be taken to refer to the number of identical residues between said sequences as determined using any standard algorithm known to those skilled in the art that maximizes the number of identical residues and minimizes the number and/or length of sequence gaps in the alignment. For example, amino acid sequence identities or similarities may be calculated using the GAP programme and/or aligned using the PILEUP programme of the Computer Genetics Group, Inc., University Research Park, Madison, Wis., United States of America. Alternatively or in addition, wherein more than two amino acid sequences are being compared, the ClustalW programme of Thompson et al (1994) can be used.

Those skilled in the art will be aware that the percentage identity to a particular sequence is related to the phylogenetic distance between the species from which the sequences are derived, and as a consequence, those sequences from species distantly-related to Vitis are likely to have functionally-equivalent MYBPA1 proteins, albeit having a low percentage identity to VvMYBPA1 at the amino acid sequence level. Such distantly-related MYBPA1 proteins may be isolated without undue experimentation using the isolation procedures described herein, and as a consequence, are clearly encompassed by the present invention.

Preferred sources of the MYBPA1 proteins of the present invention include any plant species known to produce tannins, and more particularly, catechin or epicatechin, in the seed coat, testa, pericarp, leaf, floral organ, or root. For example, preferred sources include those fodder or forage legumes, companion plants, food crops, trees, shrubs, or ornamentals selected from the group consisting of: Acacia spp., Acer spp., Actinidia spp., Aesculus spp., Agathis spp., Albizia spp., Alsophila spp., Andropogon spp., Arachis spp, Areca spp., Astelia spp., Astragalus spp., Baikiaea spp., Betula spp., Bruguiera spp., Burkea spp., Butea spp., Cadaba spp., Calliandra app, Camellia spp., Canna spp., Cassia spp,. Centroema spp, Chaenomeles spp., Cinnamomum spp., Coffee spp., Colophospermum spp., Coronillia spp., Cotoneaster spp., Crataegus spp., Cupressus spp., Cyathea spp., Cydonia spp., Cryptomeria spp., Cymbopogon spp., Cynthea dealbata, Cydonia oblonga, Dalbergia monetaria, Davallia divaricata, Desmodium spp., Dicksonia squarosa, Diheteropogon amplectens, Dioclea spp, Dolichos spp., Dorycnium rectum, Echinochloa pyramidalis, Ehrartia dura, spp., Eleusine coracana, Eragrestis spp., Erythrina spp, Eucalyptus robusta, Euclea schimperi, Eulalia villosa, Fagopyrum spp., Feijoa sellowiana, Fragaria spp., Flemingia spp, Freycinetia banksii, Geranium thunbergii, Ginkgo biloba, Glycine javanica, Gliricidia spp, Gossypium hirsutum, Grevillea spp., Guibourtia coleosperma, Hedysarum spp., Hemarthia altissima, Heteropogon contortus, Hordeum vulgare, Hyparrhenia rufa, Hypericum erectum, Hyperthelia dissolute, Indigo incarnate, Iris spp., Leptarrhena pyrolifolia, Lespediza spp., Leucaena leucocephala, Loudetia simplex, Lotonus bainesii, Lotus spp., Macrotyloma axillare, Malus spp., Manihot esculenta, Medicago sativa, Metasequoia glyptostroboides, Musa sapientum, Onobrychis spp., Ornithopus spp., Peltophorum africanum, Persea gratissima, Phaseolus atropurpureus, Phoenix canariensis, Phormium cookianum, Photinia spp., Picea glauca, Pinus spp., Podocarpus totara, Pogonarthria spp., Populus×euramericana, Prosopis cineraria, Pseudotsuga menziesii, Pterolobium stellatum, Pyrus communis, Quercus spp., Rhaphiolepsis umbellata, Rhopalostylis sapida, Rhus natalensis, Ribes spp., Robinia pseudoacacia, Rosa centifolia, Rubus spp., Salix spp., Schyzachyrium sanguineum, Sciadopitys verticillata, Sequoia sempervirens, Sequoiadendron giganteum, Sorghum bicolor, Sporobolus fimbriatus, Stiburus alopecuroides, Stylosanthos humilis, Tadehagi spp, Taxodium distichum, Themeda triandra, Trifolium spp., Triticum spp., Tsuga heterophylla, Vaccinium spp., Vicia sativa, Vitis vinifera, Watsonia pyramidata, and Zantedeschia aethiopica.

Even more preferably, the MYBPA1 protein of the invention is derived from a plant selected from the group consisting of: D. uncinatum, Medicago sativa, Medicago truncatula, Trifolium repens, Lotus corniculatus, Lotus japonicus, Nicotiana tabacum, Vitis vinifera, Camellia sinensis, Hordeum vulgare, Sorghum bicolor, Populus trichocarpa, Forsythia×intermedia, Thuja plicata, Pinus radiata, Pseudotsuga menziesii, and A. thaliana.

The seeds of any plant, or a tissue, cell or organ culture of any plant, are also preferred sources of the MYBPA1 protein.

The teaching provided herein clearly enables those skilled in the art to isolate a MYBPA1 protein of plants without undue experimentation. For example, the amino acid sequence of a Vitis MYBPA1 protein, or the amino acid sequence of a fragment thereof, can be used to design antibodies for use in the affinity purification of immunologically cross-reactive proteins from other plants. Those skilled in the art will recognize that such immunologically cross-reactive proteins are likely to be MYBPA1 proteins, particularly if peptide fragments having amino acid sequences that are not highly-conserved between MYBPA1 and other proteins are used as immunogens to elicit the production of those antibodies. Alternatively, such antibodies can be used to isolate cDNA clones that express immunologically cross-reactive proteins according to any art-recognized protocol, such as, for example, the procedure disclosed by Huynh at al. (1985), and the expressed protein subsequently isolated or purified. The isolation or purification of the expressed protein is facilitated by expressing the MYBPA1 protein as a fusion protein with a tag, such as, for example, glutathione-S-transferase, FLAG, or oligo-Histidine motifs. Alternatively, the MYBPA1 protein may be expressed as an inclusion body, or targeted to a specific organelle (e.g. a plastid, vacuole, mitochondrion, nucleus, etc) to facilitate subsequent isolation. Procedures for recombinantly-expressing proteins, and for sequestering and/or purifying recombinantly-expressed proteins, are well-known to those skilled in the art. Accordingly, the present invention is not to be limited by the mode of purification of exemplified herein.

A further aspect of the present invention provides an antibody molecule prepared by a process comprising immunizing an animal with an immunologically-effective amount of an isolated MYBPA1 protein or a fragment comprising at least about 10 contiguous amino acids in length of said MYBPA1 protein, and isolating a monoclonal or polyclonal antibody from said animal.

This aspect of the invention clearly extends to any monoclonal or polyclonal antibody that binds to a MYBPA1 protein or to a fragment comprising at least about 10 contiguous amino acids in length of said MYBPA1 protein.

The term “antibody” as used herein, is intended to include fragments thereof which are also specifically reactive with a MYBPA1 protein of the present invention, or with a fragment thereof as described herein. Antibodies can be fragmented using conventional techniques and the fragments screened for utility in the same manner as for whole antibodies. For example, F(ab′)2 fragments can be generated by treating antibody with pepsin. The resulting F(ab′)2 fragment can be treated to reduce disulfide bridges to produce Fab′ fragments.

Those skilled in the art will be aware of how to produce antibody molecules when provided with the MYBPA1 protein or a fragment thereof, according to the embodiments described herein. For example, polyclonal antisera or monoclonal antibodies can be made using standard methods. A mammal, (e.g., a mouse, hamster, or rabbit) can be immunized with an immunogenic form of the polypeptide which elicits an antibody response in the mammal. Techniques for conferring immunogenicity on a polypeptide include conjugation to carriers or other techniques well known in the art. For example, the polypeptide can be administered in the presence of adjuvant. The progress of immunization can be monitored by detection of antibody titers in plasma or serum. Standard ELISA or other immunoassay can be used with the immunogen as antigen to assess the levels of antibodies. Following immunization, antisera can be obtained and, if desired IgG molecules corresponding to the polyclonal antibodies may be isolated from the sera.

To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art. For example, the hybridoma technique originally developed by Kohler and Milstein (1975) as well as other techniques such as the human B-cell hybridoma technique (Kozbor et al., 1983), the EBV-hybridoma technique to produce human monoclonal antibodies (Cole at al., 1985), and screening of combinatorial antibody libraries (Huse et al., 1989). Hybridoma cells can be screened immunochemically for production of antibodies which are specifically reactive with the polypeptide and monoclonal antibodies isolated.

Those skilled in the art will recognize that cross-reactive proteins (i.e. proteins that bind to anti-MYBPA1 protein antibodies) are most likely to be MYBPA1 proteins. Accordingly, the antibodies described herein are useful for isolating or purifying MYBPA1 proteins from any plant, by standard procedures of affinity purification using antibodies. Alternatively, they are used for isolating nucleic acid expressing said MYBPA1 proteins, from any source, using any art-recognized procedure. Alternatively, the antibodies can be used to immunoprecitiate or inhibit MYBPA1 protein activity present in cell extracts in vitro. Alternatively, they can be used to localize MYBPA1 protein activity in cells, such as, for example, by immunohistochemical staining of plant tissue sections.

A further aspect of the present invention provides an isolated nucleic acid molecule comprising (i) a nucleotide sequence that encodes a MYB polypeptide having activity as a transcription factor in the synthesis of proanthocyanidin in plants, wherein said polypeptide activates in said plants (a) promoters of the leucoanthocyanidin (LAR) and anthocyanidin reductase (ANR) genes, and (b) promoters of at least two of the genes of the general flavonoid pathway; or (ii) a nucleotide sequence that encodes a fragment comprising at least about 10 contiguous amino acids derived from said polypeptide; or (iii) a nucleotide sequence that is complementary to (i) or (ii).

In one embodiment, said nucleotide sequence (i) is a sequence that encodes a polypeptide which comprises an amino acid sequence substantially corresponding the to VvMYBPA1 protein sequence set forth in SEQ ID NO: 2 or an orthologue or homologue thereof, or an amino acid sequence having at least 40% identity overall thereto, or an amino acid sequence having at least 40% identity of amino acids 116-286 of said VvMYBPA1 protein sequence.

The isolated nucleic acid molecule of the invention can be derived from any plant species. The present invention is not to be limited by the species origin of nucleic acid encoding the MYBPA1 protein. Without limiting the scope of the invention, preferred plant sources include those plants referred to in the index to the International Code of Botanical Nomenclature (Tokyo Code) as adopted by the Fifteenth International Botanical Congress, Yokohama, August-September 1993 (published as International Code of Botanical Nomenclature (Tokyo Code) Regnum Vegetabile 131, Koeltz Scientific Books, Königstein, ISBN 3-87429-367-X or 1-878762-66-4 or 80-901699-1-0). More preferably, the isolated nucleic acid of the invention is derived from a plant listed supra.

Even more preferably, the nucleic acid of the invention is derived from a plant selected from the group consisting of: D. uncinatum, Medicago sativa, Medicago truncatula, Trifolium repens, Lotus corniculatus, Lotus japonicus, Nicotiana tabacum, Vitis vinifera, Camellia sinensis, Hordeum vulgare, Sorghum bicolor, Populus trichocarpa, Forsythia×intermedia, Thuja plicata, Pinus radiata, Pseudotsuga menziesii, and A. thaliana.

The nucleic acid of the invention may be in the form of RNA or DNA, such as, for example, single-stranded, double-stranded or partially double-stranded cDNA, genomic DNA, oligonucleotides, or DNA amplified by polymerase chain reaction (PCR); or a mixed polymer comprising RNA and DNA.

Preferably, the percentage identity to an amino acid sequence presented herein is at least about 50%, more preferably at least about 60%, even more preferably at least about 70%, and still even more preferably at least about 80% or at least about 90%.

Nucleic acid of the present invention may be derived by organic synthesis based upon the nucleotide sequence of a naturally-occurring MYBPA1 gene, or from a MYBPA1 gene per se. Reference herein to a “MYBPA1 gene” is to be taken in its broadest context and includes a member selected from the group consisting of:
(i) a classical genomic gene encoding all or part of a MYBPA1 protein, and consisting of transcriptional and/or translational regulatory sequences and/or a coding region and/or untranslated sequences (i.e. introns, 5′- and 3′-untranslated sequences);
(ii) mRNA or cDNA encoding all or part of a MYBPA1 protein, said mRNA or cDNA corresponding to the coding regions (i.e. exons) and 5′- and 3′-untranslated sequences of the genomic gene;
(iii) a synthetic or fusion molecule encoding all or part of a MYBPA1 protein; and
(iv) a complementary nucleotide sequence to any one of (i) to (iii).

Preferred MYBPA1 genes of the present invention are derived from naturally-occurring sources using standard recombinant techniques, such as, for example, mutagenesis, to introduce single or multiple nucleotide substitutions, deletions and/or additions relative to the wild-type sequence.

It is clearly within the scope of the present invention to include any nucleic acid comprising a nucleotide sequence complementary to a MYBPA1 gene as defined herein, in particular complementary nucleotide sequences that are useful as hybridization probes, or amplification primers, for isolating or identifying a MYBPA1 gene, or for reducing the level of expression of an endogenous MYBPA1 gene in a cell, tissue, organ, or whole plant. Such complementary nucleotide sequences may be in the form of RNA, such as, for example, antisense mRNA, or a ribozyme; DNA, such as, for example, single-stranded or double-stranded cDNA, genomic DNA, single-stranded or double-stranded synthetic oligonucleotides, or DNA amplified by polymerase chain reaction (PCR); or a mixed polymer comprising RNA and DNA. As will be known to those skilled in the art, sequences complementary to the coding region and/or non-coding region of a gene may be useful for such applications.

An antisense molecule is nucleic acid comprising a nucleotide sequence that is complementary to mRNA, or a DNA strand, that encodes protein, albeit not restricted to sequence having complementarity to the protein-encoding region. Preferred antisense molecules comprise RNA capable of hybridizing to mRNA encoding all or part of a MYBPA1 protein. Antisense molecules are thought to interfere with the translation or processing or stability of the mRNA of the target gene, thereby inactivating its expression. Methods of devising antisense sequences are well known in the art and examples of these are can be found in U.S. Pat. No. 5,190,131, European patent specification 0467349-A1, European patent specification 0223399-A1 and European patent specification 0240208, which are incorporated herein by reference. The use of antisense techniques in plants has been reviewed by Bourque (1995) and Senior (1998). Bourque lists a large number of examples of how antisense sequences have been utilized in plant systems as a method of gene inactivation. She also states that attaining 100% inhibition of any enzyme activity may not be necessary as partial inhibition will more than likely result in measurable change in the system. Senior (1998) states that antisense methods are now a very well established technique for manipulating gene expression.

Antisense molecules for MYBPA1 genes can be based on the Arabidopsis mRNA sequences or based on homologies with DNA or mRNA sequences derived from other species, for example white clover These antisense sequences may correspond to the structural genes or for sequences that effect control over the gene expression or splicing event. For example, the antisense sequence may correspond to the targeted coding region of the gene or to the 5′-untranslated region (UTR) or the 3′-UTR or combination of these. It may be complementary in part to intron sequences, which may be spliced out during or after transcription, preferably only to exon sequences of the target gene. In view of the generally greater divergence of the UTRS, targeting these regions provides greater specificity of gene inhibition. The length of the antisense sequence should be at least 19 contiguous nucleotides, preferably at least 30 or 50 nucleotides, and more preferably at least 100, 200, 500 or 1000 nucleotides. The full-length sequence complementary to the entire gene transcript may be used. The length is most preferably 100-2000 nucleotides. The degree of homology of the antisense sequence to the targeted transcript should be at least 85%, preferably at least 90% and more preferably 95-100%. The antisense RNA molecule may of course comprise unrelated sequences which may function to stabilize the molecule.

In the present context, a “ribozyme” is a synthetic RNA molecule which comprises one or preferably two hybridizing sequences, each of at least about 5-20 contiguous nucleotides in length, capable of hybridizing to mRNA encoding a MYBPA1 protein, and possessing an endoribonuclease activity that is capable of catalytically cleaving said mRNA. Ribozymes can cleave the mRNA molecules at specific sites defined by the hybridizing sequences. The cleavage of the RNA inactivates the expression of the target gene. The ribozymes may also act as an antisense molecule, which may contribute to the gene inactivation. The ribozymes contain one or more catalytic domains, preferably of the hammerhead or hairpin type, between the hybridizing sequences. Other ribozyme motifs may be used including RNAseP, Group I or II introns, and hepatitis delta virus types. Reference is made to European patent specification 0321201 and U.S. Pat. No. 6,221,661. The use of ribozymes to inactivate genes in transgenic plants has been demonstrated. As with antisense molecules, ribozymes may target regions in the mRNA other than those of the protein-encoding region, such as, for example, in the untranslated region of a MYBPA1 gene.

The term “untranslated region” in this context means a region of a genomic gene or cDNA that is normally transcribed in a cell but not translated into an amino acid sequence of a MYBPA1 protein. Accordingly, the term “untranslated region” includes nucleic acid comprising a nucleotide sequence derived from the 5′-end of mRNA to immediately preceding the ATG translation start codon; nucleic acid comprising a nucleotide sequence from the translation stop codon to the 3′-end of mRNA; and any intron sequence that is cleaved from a primary mRNA transcript during mRNA processing.

The present invention further encompasses within its scope nucleic acid molecules comprising a first sense nucleotide sequence derived from mRNA, or a DNA strand, encoding a MYBPA1 protein, and a second antisense nucleotide sequence complementary to mRNA encoding a MYBPA1 protein, such as for example, in the form of a post-transcription gene silencing (PTGS) molecule. The first and second sequences may be linked in head-to-head or tail-to-tail (inverted) configuration. As with antisense molecules or ribozymes, such molecules need not be derived exclusively from the open reading frame of a MYBPA1 gene. Sequences derived from untranslated regions, in particular the 5′ or 3′ untranslated regions, may be preferred for the sense nucleotide sequence. Preferred PTGS molecules will have a region of self-complementarity and be capable of forming a hairpin loop structure, such as those described in International Patent Application No. PCT/IB99/00606. Whilst not being bound by any theory or mode of action, a PTGS molecule has the potential to sequester sense MYBPA1-encoding mRNA in a cell, such that the sequestered mRNA is degraded. In a preferred embodiment, the sense and antisense sequences are separated by a spacer region that comprises an intron which, when transcribed into DNA, is spliced out. This arrangement has been shown to result in a higher efficiency of gene silencing (Smith et al., 2000). The double-stranded RNA region may comprise one or two or more RNA molecules, transcribed from either one DNA region or two or more. The presence of the double stranded molecule is thought to trigger a response from an endogenous plant system that destroys both the double stranded RNA and also the homologous RNA transcript from the target plant gene, efficiently reducing or eliminating the activity of the target gene. The length of the sense and antisense sequences that hybridise should each be at least 19 contiguous nucleotides, preferably at least 30 or 50 nucleotides, and more preferably at least 100, 200, 500 or 1000 nucleotides. The full-length sequence corresponding to the entire gene transcript may be used. The lengths are most preferably 100-2000 nucleotides. The degree of homology of the sense and antisense sequences to the targeted transcript should be at least 85%, preferably at least 90% and more preferably 95-100%. The RNA molecule may of course comprise unrelated sequences which may function to stabilize the molecule. The RNA molecule may be expressed under the control of a RNA polymerase II or RNA polymerase III promoter. Examples of the latter include tRNA or snRNA promoters such as a U6 promoter.

The antisense, cosuppression or double stranded RNA molecules may also comprise a largely double-stranded RNA region, preferably comprising a nuclear localization signal, as described in PCT/AU03/00292. In a preferred embodiment, the largely double-stranded region is derived from a PSTVd type viroid or comprises at least 35 CUD trinucleotide repeats.

Preferred nucleic acid encoding a MYBPA1 protein will be in the form of sense nucleic acid. In the present context, the term “sense nucleic acid” shall be taken to mean RNA or DNA comprising a nucleotide sequence derived from the strand of DNA or RNA that encodes a full-length MYBPA1 protein, or a part thereof, including both coding and non-coding sequences. As will be known to those skilled in the art, sense nucleic acid may be used to for the purposes of ectopically expressing mRNA, or protein, in a cell, or alternatively, to down-regulate expression (e.g. co-suppression), or to identify or isolate a MYBPA1 gene, or to identify or isolate complementary sequences, such as, for example, antisense mRNA. As will be known to those skilled in the art, “co-suppression” is the reduction in expression of an endogenous gene that occurs when one or more copies of said gene, or one or more copies of a substantially similar gene, or fragments thereof, are introduced into the cell. The mechanism of co-suppression is not well understood but is thought to involve post-transcriptional gene silencing (PTGS) and in that regard may be very similar to many examples of antisense suppression or duplex RNA suppression. It involves introducing an extra copy of a gene or a fragment thereof into a plant in the sense orientation with respect to a promoter for its expression. The size of the sense fragment, its correspondence to target gene regions, and its degree of homology to the target gene are as for the antisense sequences described above. In some instances the additional copy of the gene sequence interferes with the expression of the target plant gene. Reference is made to Patent specification WO 97/20936 and European patent specification 0465572 for methods of implementing co-suppression approaches. As will be known to those skilled in the art, whilst the coding region of a gene is required to ectopically-express protein in a cell, the coding region and/or non-coding region of a gene may be useful for other applications referred to herein.

Sense nucleic acid molecules will preferably comprise the full-length open reading frame of an endogenous MYBPA1 gene, however may be less than full-length. It will be apparent from the definition of the term “MYBPA1 gene” provided herein above, that the present invention encompasses within its scope any nucleic acid fragment of the full-length open reading frame of a MYBPA1 gene, that is at least useful as a hybridization probe or amplification primer for isolating a MYBPA1 gene, or for modifying the level of expression of an endogenous MYBPA1 gene.

Preferred fragments of a MYBPA1 gene of the invention, for isolating or identifying homologous genes in the same or another species, are derived from the open reading frame. In the present context, an “open reading frame” is any nucleotide sequence encoding an amino acid sequence of a MYBPA1 protein, and preferably, at least about 10 contiguous amino acids of a MYBPA1 protein.

As will be known to those skilled in the art, where homologous MYBPA1 gene sequences are from divergent species to the species from which the fragment is derived, fragments of at least about 20 nucleotides in length from within the open reading frame of the MYBPA1 gene, more preferably at least about 30-50 nucleotides in length, and more preferably at least about 100 nucleotides in length, or 500 nucleotides in length, are preferred.

In the case of fragments for isolating or identifying an identical target MYBPA1 gene, or a MYBPA1 gene from a closely-related species, the fragment may be derived from any part of a known MYBPA1 gene, such as, for example, from the open reading frame, an untranslated region, or an intron, or promoter sequence.

In the present context, the term “promoter” means a nucleotide sequence comprising a transcriptional regulatory sequence for initiation of transcription, such as, for example, the TATA box which is required for accurate transcription initiation, with or without a CCAAT box sequence and additional cis-acting regulatory elements (i.e. upstream activating sequences, enhancers and silencers). Preferred promoters are those derived from a MYBPA1 gene, or those that may alter MYBPA1 gene expression in response to developmental and/or external stimuli, or in a tissue-specific manner.

The present invention also provides an isolated nucleic acid molecule comprising a nucleotide sequence selected from the group consisting of: (i) a nucleotide sequence having at least about 40% identity overall to the VvMYBPA1 nucleotide sequence set forth in SEQ ID NO: 1 or a protein coding region thereof; (ii) a nucleotide sequence that is complementary to (i); or (iii) a nucleotide sequence that hybridises to at least about 20 contiguous nucleotides of (i) or (ii) under at least low stringency conditions, preferably under moderate stringency conditions and more preferably under high stringency conditions.

Preferably, the percentage identity to a nucleotide sequence presented herein is at least about 50%, more preferably at least about 60%, even more preferably at least about 70%, and even more preferably, at least about 80%, and still even more preferably at least about 90%. In preferred embodiments, the invention provides nucleotide sequences which have at least 40%, 50%, 60%, 70%, 80% or even 90% nucleotide sequence identity to the coding region of the VvMYBPA1 nucleotide sequence.

For the purposes of defining the level of stringency in a hybridization to any one of the nucleotide sequences disclosed herein, a low stringency hybridization may comprise a hybridization and/or a wash carried out using a salt concentration equivalent to SSC buffer in the range of 2×SSC to 6×SSC buffer; a detergent concentration in the range of 0.1% (w/v) SDS to 1% (w/v) SDS; and a temperature in the range of between ambient temperature to about 42° C. Those skilled in the art will be aware that several different hybridization conditions may be employed. For example, Church buffer may be used at a temperature in the range of between ambient temperature to about 45° C.

Preferably, the stringency of hybridization is at least moderate stringency, even more preferably at high stringency. Generally, the stringency is increased by reducing the concentration of SSC buffer, and/or increasing the concentration of SDS in the hybridization buffer or wash buffer and/or increasing the temperature at which the hybridization and/or wash are performed. Conditions for hybridizations and washes are well understood by one normally skilled in the art. For example, a moderate stringency hybridisation may comprise a hybridization and/or wash carried out using a salt concentration in the range of between about 1×SSC buffer and 2×SSC buffer; a detergent concentration of up to about 0.1% (w/v) SDS; and a temperature in the range of about 45° C. to 55° C. Alternatively, Church buffer may be used at a temperature of about 55° C., to achieve a moderate stringency hybridization. A high stringency hybridisation may comprise a hybridization and/or wash using a salt concentration in the range of between about 0.1×SSC buffer and about 1×SSC buffer; a detergent concentration of about 0.1% (w/v) SDS; and a temperature of about 55° C. to about 65° C., or alternatively, a Church Buffer at a temperature of at least 65° C. Variations of these conditions will be known to those skilled in the art.

Clarification of the parameters affecting hybridization between nucleic acid molecules, is provided by Ausubel et al. (1987).

Although the present inventors have successfully isolated the MYBPA1 gene using oligonucleotide primers of only about 20 nucleotides in length, those skilled in the art will recognize that the specificity of hybridization increases using longer probes, or primers, to detect genes in standard hybridization and PCR protocols. Such approaches are facilitated by the provision herein of full-length cDNAs from a number of diverse species. For example, persons skilled in the art are readily capable of aligning the nucleotide sequences or amino acid sequences provided herein to identify conserved regions thereof, to facilitate the identification of sequences from other species or organisms. For example, conserved regions of the MYBPA1 protein may facilitate the preparation of a hybridization probe, or primer, comprising at least about 30 nucleotides in length. Accordingly, preferred nucleotide sequences according to this embodiment of the invention will hybridize to at least about 30 contiguous nucleotides, more preferably at least about 50 contiguous nucleotides, even more preferably at least about 100 contiguous nucleotides, and still even more preferably at least about 500 contiguous nucleotides.

In a particularly preferred embodiment, the nucleic acid of the invention comprises the sequence set forth as the VvMYBPA1 nucleotide sequence, a protein coding region thereof, or a sequence complementary thereto.

The present invention clearly encompasses within its scope those nucleic acid molecules from organisms other than those plants specifically described herein that encode MYBPA1 proteins, and have sequence homology to the exemplified sequences of the invention. Accordingly, in a further embodiment, the present invention provides an isolated nucleic acid molecule comprising a nucleotide sequence that encodes a MYBPA1 protein or a fragment thereof, wherein said nucleic acid molecule is isolated by a process comprising:
(i) hybridizing a probe or primer comprising at least about 20 contiguous nucleotides of the VvMYBPA1 nucleotide sequence or a degenerate or complementary nucleotide sequence thereto, to nucleic acid of plants;
(ii) detecting said hybridization;
(iii) isolating the hybridized nucleic acid; and
(iv) determining the amino acid sequence encoded by the hybridized nucleic acid or the function of said amino acid sequence so as to determine that the hybridized nucleic acid encodes said MYBPA1 protein.

The use of probes or primers encoding fragments of the VvMYBPA1 amino acid sequence are also contemplated herein, the only requirement being that such probes or primers are capable of hybridizing to a MYBPA1 gene.

The related sequence being identified may be present in a gene library, such as, for example, a cDNA or genomic gene library.

The library may be any library capable of maintaining nucleic acid of eukaryotes, such as, for example, a BAC library, YAC library, cosmid library, bacteriophage library, genomic gene library, or a cDNA library. Methods for the production, maintenance, and screening of such libraries with nucleic acid probes or primers, or alternatively, with antibodies, are well known to those skilled in the art. The sequences of the library are usually in a recombinant form, such as, for example, a cDNA contained in a virus vector, bacteriophage vector, yeast vector, baculovirus vector, or bacterial vector. Furthermore, such vectors are generally maintained in appropriate cellular contents of virus hosts.

In particular, cDNA may be contacted, under at least low stringency hybridization conditions or equivalent, with a hybridization-effective amount of a probe or primer.

In one embodiment, the detection means is a reporter molecule capable of giving an identifiable signal (e.g. a radioisotope such as ³²P or ³⁵S or a biotinylated molecule) covalently linked to the isolated nucleic acid molecule of the invention.

Conventional nucleic acid hybridization reactions, such as, for example, those described by Ausubel at al., are encompassed by the use of such detection means.

In an alternative method, the detection means is any known format of the polymerase chain reaction (PCR). According to this method, degenerate pools of nucleic acid “primer molecules” of about 20-50 nucleotides in length are designed based upon any one or more of the nucleotide sequences disclosed herein, or a complementary sequence thereto. In one approach related sequences (i.e. the “template molecule”) are hybridized to two of said primer molecules, such that a first primer hybridizes to a region on one strand of the double-stranded template molecule and a second primer hybridizes to the other strand of said template, wherein the first and second primers are not hybridized within the same or overlapping regions of the template molecule and wherein each primer is positioned in a 5′- to 3′-orientation relative to the position at which the other primer is hybridized on the opposite strand. Specific nucleic acid molecule copies of the template molecule are amplified enzymatically, in a polymerase chain reaction (PCR), a technique that is well known to one skilled in the art. McPherson et al (1991) describes several formats of PCR.

The primer molecules may comprise any naturally occurring nucleotide residue (i.e. adenine, cytidine, guanine, and thymidine) and/or comprise inosine or functional analogues or derivatives thereof, capable of being incorporated into a polynucleotide molecule. The nucleic acid primer molecules may also be contained in an aqueous mixture of other nucleic acid primer molecules or be in a substantially pure form.

Preferably, the sequence detected according to this embodiment originates from a plant as listed supra.

The present invention clearly extends to any synthetic or chimeric gene constructs that comprise the isolated nucleic acid molecule of the present invention, such as, for example, any expression gene constructs produced for expressing said nucleic acid molecule in a bacterial, insect, yeast, plant, fungal, or animal cell.

Accordingly, a further aspect of the present invention is directed to a synthetic or chimeric gene construct comprising an isolated nucleic acid that encodes a MYBPA1 protein or a biologically active fragment thereof (i.e., a fragment of a MYBPA1 protein having the biological activity of the MYBPA1 protein), or complementary nucleotide sequence thereto. The invention also provides a gene construct encoding an inhibitory molecule such as, for example, an antisense, ribozyme, PTGS or co-suppression molecule that is capable of inhibiting MYBPA1 gene activity in a cell. In a preferred embodiment, the invention provides a chimeric gene construct in which the coding region encoding a MYBPA1 protein or a biologically active fragment thereof is capable of being expressed from a promoter that does not naturally control expression of the MYBPA1 protein (heterologous promoter).

Those skilled in the art will also be aware that expression of a MYBPA1 gene, or a complementary sequence thereto, in a cell, requires said gene to be placed in operable connection with a promoter sequence. The choice of promoter for the present purpose may vary depending upon the level of expression required and/or the tissue, organ and species in which expression is to occur.

References herein to placing a nucleic acid molecule under the regulatory control of a promoter sequence mean positioning said molecule such that expression is controlled by the promoter sequence. A promoter is usually, but not necessarily, positioned upstream, or at the 5′-end, of the nucleic acid molecule it regulates. Furthermore, the regulatory elements comprising a promoter are usually positioned within 2 kb of the start site of transcription of the gene. In the construction of heterologous promoter/structural gene combinations, it is generally preferred to position the promoter at a distance from the gene transcription start site that is approximately the same as the distance between that promoter and the gene it controls in its natural setting (i.e., the gene from which the promoter is derived). As is known in the art, some variation in this distance can be accommodated without loss of promoter function. Similarly, the preferred positioning of a regulatory sequence element with respect to a heterologous gene to be placed under its control is defined by the positioning of the element in its natural setting (i.e., the gene from which it is derived). Again, as is known in the art, some variation in this distance can also occur.

Examples of promoters suitable for use in gene constructs of the present invention include promoters derived from the genes of viruses, yeast, moulds, bacteria, insects, birds, mammals and plants, preferably those capable of functioning in isolated yeast or plant cells. The promoter may regulate expression constitutively, or differentially, with respect to the tissue in which expression occurs. Alternatively, expression may be differential with respect to the developmental stage at which expression occurs, or in response to external stimuli such as physiological stresses, or temperature.

Examples of promoters useful for expression in plants include the CaMV 35S promoter, NOS promoter, octopine synthase (OCS) promoter, Arabidopsis thaliana SSU gene promoter, the meristem-specific promoter (meri1), napin seed-specific promoter, actin promoter sequence, sub-clover stunt virus promoters (International Patent Application No. PCT/AU95/00552), and the like. In addition to the specific promoters identified herein, cellular promoters for so-called housekeeping genes are useful. Promoters derived from genomic gene equivalents of the cDNAs described herein are particularly contemplated for regulating expression of MYBPA1 genes, or complementary sequences thereto, in plants. Inducible promoters, such as, for example, a heat shock-inducible promoter, heavy metal-inducible promoter (e.g. metallotheinin gene promoter), ethanol-inducible promoter, or stress-inducible promoter, may also be used to regulate expression of the introduced nucleic acid of the invention under specific environmental conditions.

For certain applications, it is preferable to express the MYBPA1 gene of the invention specifically in particular tissues of a plant, such as, for example, to avoid any pleiotropic effects that may be associated with expressing said gene throughout the plant. In particular, the MYBPA1 gene may be ectopically expressed in a tissue-specific manner in parts or tissues of the plant in which the gene is not expressed in wild type plants, for example in the leaves or stems or seeds or storage organs of the plant. As will be known to the skilled artisan, tissue-specific or cell-specific promoter sequences (such as promoters that are expressed preferentially in epidermal cells) may be required for such applications. For expression in particular plant tissues, reference is made to the publicly available or readily available sources of promoter sequences known to those skilled in the art.

For expression in yeast or bacterial cells, it is preferred that the promoter is selected from the group consisting of: GAL1, GAL10, CYC1, CUP1, PGK1, ADH2, PH05, PRB1, GUT1, SP013, ADH1, CMV, SV40, LACZ, T3, SP6, T5, and T7 promoter sequences.

The gene construct may further comprise a terminator sequence and be introduced into a suitable host cell where it is capable of being expressed to produce a recombinant dominant-negative polypeptide gene product or alternatively, a co-suppression molecule, a ribozyme, gene silencing or antisense molecule.

The term “terminator” refers to a DNA sequence at the end of a transcriptional unit which signals termination of transcription. Terminators are 3′-non-translated DNA sequences containing a polyadenylation signal, which facilitates the addition of poly(A) sequences to the 3′-end of a primary transcript.

Terminators active in cells derived from viruses, yeast, moulds, bacteria, insects, birds, mammals and plants are known and described in the literature. They may be isolated from bacteria, fungi, viruses, animals and/or plants.

Examples of terminators particularly suitable for use in the gene constructs of the present invention include the nopaline synthase (NOS) gene terminator of Agrobacterium tumefaciens, the terminator of the Cauliflower mosaic virus (CaMV) 35S gene, the zein gene terminator from Zea mays, the Rubisco small subunit (SSU) gene terminator sequences, subclover stunt virus (SCSV) gene sequence terminators (International Patent Application No. PCT/AU95/00552), and the terminator of the Flayeria bidentis malic enzyme gene meA3 (International Patent Application No. PCT/AU95/00552).

Those skilled in the art will be aware of additional promoter sequences and terminator sequences suitable for use in performing the invention. Such sequences may readily be used without any undue experimentation.

The gene constructs of the invention may further include an origin of replication sequence which is required for replication in a specific cell type, for example a bacterial cell, when said gene construct is required to be maintained as an episomal genetic element (e.g. plasmid or cosmid molecule) in said cell.

Preferred origins of replication for use in bacterial cells include, but are not limited to, the f1-ori and colE1 origins of replication. The 2-micron origin of replication may be used in gene constructs for use in yeast cells.

The gene construct may further comprise a selectable marker gene or genes that are functional in a cell into which said gene construct is introduced. As used herein, the term “selectable marker gene” includes any gene which confers a phenotype on a cell in which it is expressed to facilitate the identification and/or selection of cells which are transfected or transformed with a gene construct of the invention or a derivative thereof.

Suitable selectable marker genes contemplated herein include the ampicillin resistance (Amp^{r}), tetracycline resistance gene (Tc^{r}), bacterial kanamycin resistance gene (Kan^{r}), phosphinothricin resistance gene, neomycin phosphotransferase gene (nptII), hygromycin resistance gene, β-glucuronidase (GUS) gene, chloramphenicol acetyltransferase (CAT) gene and luciferase gene, amongst others.

In a preferred embodiment of the invention, the gene construct is a binary gene construct, more preferably a binary gene construct comprising a selectable marker gene selected from the group consisting of: bar, nptII and spectinomycin resistance genes. Those skilled in the art will be aware of the chemical compounds to which such selectable marker genes confer resistance.

In an even more preferred embodiment, the binary construct comprises the Streptomyces hygroscopicus bar gene, placed operably in connection with the CaMV 35S promoter sequence. Still more preferably, the binary construct comprises the Streptomyces hygroscopicus bar gene, placed operably in connection with the CaMV 35S promoter sequence and upstream of the terminator sequence of the octopine synthase (ocs) gene.

A further aspect of the invention contemplates a cell comprising a non-endogenous MYBPA1 gene, preferably wherein said MYBPA1 gene is present in said cell in an expressible format.

As used herein, the word “cell” shall be taken to include an isolated cell, or a cell contained within organized tissue, a plant organ, or whole plant.

Preferably the cell is a bacterial cell, such as, for example, E. coli or A. turnefaciens, or a plant cell, such as a legume, more particularly a fodder or forage legume such as Medicago spp. and Trifolium spp. Even more preferably, the cell is an Agrobacterium tumefaciens strain carrying a disarmed Ti plasmid, such as, for example, the Agrobacterium tumefaciens strain is designated AGL1 (Lazo et al., 1991). However, as will be understood by those skilled in the art, the isolated nucleic acid of the present invention may be introduced to any cell and maintained or replicated therein, for the purposes of generating probes or primers, or to produce recombinant MYBPA1 protein, or a fragment thereof. Accordingly, the present invention is not limited by the nature of the cell.

Those skilled in the art will be aware that whole plants may be regenerated from individual transformed cells. Accordingly, the present invention also extends to any plant material which comprises a gene construct according to any of the foregoing embodiments or expresses a sense, antisense, ribozyme, PTGS or co-suppression molecule, and to any cell, tissue, organ, plantlet or whole plant derived from said material.

A further aspect of the invention contemplates a transformed plant comprising a non-endogenous MYBPA1 gene or fragment thereof introduced into its genome, or a nucleotide sequence that is complementary to said MYBPA1 gene or said fragment, in an expressible format. The term “non-endogenous MYBPA1 gene” includes genes in which a MYBPA1 coding region that is endogenous to the plant is operably under the control of a non-endogenous promoter.

The term “endogenous” as used herein refers to the normal complement of a stated integer which occurs in an organism in its natural setting or native context (i.e. in the absence of any human intervention, in particular any genetic manipulation).

The term “non-endogenous” as used herein shall be taken to indicate that the stated integer is derived from a source which is different to the plant material, plant cell, tissue, organ, plantlet or whole plant into which it has been introduced. The term “non-endogenous” shall also be taken to include a situation where genetic material from a particular species is introduced, in any form, into an organism belonging to the same species as an addition to the normal complement of genetic material of that organism.

Preferably, the transformed plant of the invention further expresses a non-endogenous MYBPA1 protein. Such expression may be ectopic expression in cells or tissues of the transformed plant in which the protein is not usually expressed. This aspect of the invention clearly extends to any plant parts, plant material, cells, tissues, organs or plantlets, or progeny plants, that are derived from the primary transformed plant.

Preferably, the plant part, plant material, plant cell, tissue, organ, plantlet or whole plant comprises or is derived from a fodder crop, companion plant, food crop, fruit, tree, shrub or ornamental plant as described herein, or a tissue, cell or organ culture of any of said plants or the seeds of any of said plants, in particular a legume, more particularly a fodder and forage legume such as Medicago spp. and Trifolium spp., or a food crop or fruit, more particularly a Vitis spp.

The present invention extends to the progeny and clonal derivatives of a plant according to any one of the embodiments described herein.

As will be known those skilled in the art, transformed plants are generally produced by introducing a gene construct, or vector, into a plant cell, by transformation or transfection means. The isolated nucleic acid molecule of the invention, especially the MYBPA1 gene of the invention, or a gene construct comprising same, is introduced into a cell using any known method for the transfection or transformation of a plant cell. Wherein a cell is transformed by the gene construct of the invention, a whole plant may be regenerated from a single transformed cell, using methods known to those skilled in the art.

By “transfect” is meant that the MYBPA1 gene or a PTGS molecule, antisense molecule, co-suppression molecule, or ribozyme comprising sequences derived from the MYBPA1 gene, is introduced into a cell without integration into the cell's genome. Alternatively, a gene construct comprising said gene, said molecule, or said ribozyme, placed operably under the control of a suitable promoter sequence, can be used.

By “transform” is meant the MYBPA1 gene or a PTGS molecule, antisense molecule, co-suppression molecule, or ribozyme comprising sequences derived from the MYBPA1 gene, is introduced into a cell and integrated into the genome of the cell. Alternatively, a gene construct comprising said, gene, said molecule, or said ribozyme, placed operably under the control of a suitable promoter sequence, can be used.

Means for introducing recombinant DNA into plant cells or tissue include, but are not limited to, direct DNA uptake into protoplasts, PEG-mediated uptake to protoplasts, electroporation, microinjection of DNA, microparticle bombardment of tissue explants or cells, vacuum-infiltration of tissue with nucleic acid, and T-DNA-mediated transfer from Agrobacterium to the plant tissue. All of these techniques are well known in the art.

For example, transformed plants can be produced by the method of in planta transformation method using Agrobacterium tumefaciens, wherein A. tumefaciens is applied to the outside of the developing flower bud and the binary vector DNA is then introduced to the developing microspore and/or macrospore and/or the developing seed, so as to produce a transformed seed. Those skilled in the art will be aware that the selection of tissue for use in such a procedure may vary, however it is preferable generally to use plant material at the zygote formation stage for in plants transformation procedures.

A method for the efficient introduction of genetic material into Trifolium repens and regeneration of whole plants therefrom is also described in International Patent Application No. PCT/AU97/00529, Voisey et al (1994), or Larkin et al., (1996).

Alternatively, microparticle bombardment of cells or tissues may be used, particularly in cases where plant cells are not amenable to transformation mediated by A. tumefaciens. In such procedures, microparticle is propelled into a cell to produce a transformed cell. Any suitable biolistic cell transformation methodology and apparatus can be used in performing the present invention. Stomp et al. (U.S. Pat. No. 5,122,466) or Sanford and Wolf (U.S. Pat. No. 4,945,050) discloses exemplary apparatus and procedures. When using biolistic transformation procedures, the genetic construct may incorporate a plasmid capable of replicating in the cell to be transformed. Exemplary microparticles suitable for use in such systems include 1 to 5 micron gold spheres. The DNA construct may be deposited on the microparticle by any suitable technique, such as by precipitation.

A whole plant may be regenerated from the transformed or transfected cell, in accordance with procedures well known in the art. Plant tissue capable of subsequent clonal propagation, whether by organogenesis or embryogenesis, may be transformed with a gene construct of the present invention and a whole plant regenerated therefrom. The particular tissue chosen will vary depending on the clonal propagation systems available for, and best suited to, the particular species being transformed. Exemplary tissue targets include leaf disks, pollen, embryos, immature embryos, scutellum, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem).

The term “organogenesis”, as used herein means a process by which shoots and roots are developed sequentially from a meristematic center.

The term “embryogenesis”, as used herein, means a process by which shoots and roots develop together in a concerted fashion (not sequentially), whether from somatic cells or gametes.

The generated transformed plants may be propagated by a variety of means, such as by clonal propagation or classical breeding techniques. For example, a first generation (or T1) transformed plant may be selfed to give homozygous second generation (or T2) transformant and the T2 plants further propagated through classical breeding techniques.

The generated transformed organisms contemplated herein may take a variety of forms. For example, they may be chimeras of transformed cells and non-transformed cells; clonal transformants (e.g., all cells transformed to contain the expression cassette), grafts of transformed and untransformed tissues (e.g., in plants, a transformed rootstock grafted to an untransformed scion).

The nucleic acid of the invention, and gene constructs comprising same, are particularly useful for modifying levels of PAs in plants. In this respect, the isolated nucleic acid of the invention placed in either the sense or the antisense orientation relative to a suitable promoter sequence, wherein said orientation will depend upon the desired end-result for which the gene construct is intended. The levels of PAs in the plant may be increased or decreased, in parts of the plant or throughout the plant, or increased in at least one tissue and decreased in at least one other tissue, for example increased in the aerial growing parts of a plant but decreased in seed.

Such plants may exhibit a range of desired traits including, but not limited to improved bloat-safety for animals grazing thereupon (i.e. less propensity to induce bloating when ingested), increased efficiency of protein utilization in ruminants with concomitant higher productivity, improved disease- or pest-resistance.

As used herein, “higher productivity” shall be taken to refer to increased production in any biological product or secondary metabolite of an animal species, in particular a livestock animal selected from the list comprising sheep, goats, alpaca, cattle, dairy cattle, amongst others, which is at least partly attributable to said animal being grazed upon or otherwise fed a plant comprising a gene construct of the present invention. Preferably, higher productivity includes increased milk yield, increased meat production or increased wool production.

Food plants comprising higher levels of PAs, which have been produced using the gene constructs of the present invention, afford the benefit of having a longer shelf life than otherwise. Whilst not being bound by any theory or mode of action, the longer shelf life of such food plants is due to the antioxidant and antimicrobial properties of PAs. These effects also provide for the development of new and improved health foods or other foodstuffs with improved anti-oxidant activities and free radical scavenging properties, which are useful in the treatment or prevention of a range of diseases.

For example, the introduction of additional copies of a MYBPA1 gene, in the sense orientation, and under the control of a strong promoter, is useful for the production of plants, in particular fodder and forage legumes and food plants, which exhibit increased PA content or more rapid rates of PA biosynthesis.

Alternatively, gene constructs comprising an MYBPA1 gene in the sense orientation may be used to complement the existing range of proanthocyanidin genes present in a plant, thereby altering the composition or timing of deposition of PAs. In a preferred embodiment, the proanthocyanidin gene from one plant species is used to transform a plant of a different species, thereby introducing novel proanthocyanidin biosynthetic metabolism to the second-mentioned plant species.

Furthermore, the gene constructs of the invention which express an active MYBPA1 protein may be introduced into non-legume companion species which serve as companion plants for bloat-inducing fodder and forage legumes such as lucerne (alfalfa) or white clover. In this embodiment, when the levels of PAs in the companion species are sufficiently high, the bloat-safe companion species counters the action of the bloat-inducing forage-legume when both crops are ingested by a grazing animal. Preferred companion plants include, but are not limited to several species of Lolium, in particular L. perenne.

In a further embodiment, the rate of PA deposition may be reduced leading to a reduction in the total tannin content of plants by transferring one or more antisense, ribozyme, PTGS, or co-suppression molecules into a plant using a suitable gene construct as a delivery system.

The benefits to be derived from reducing tannin content in plants are especially apparent in fodder crops such as, but not limited to Onobrychis viciifolia, Onithopus pinnatus, Ornithpus compressus, Coronilla varia, Lotus corniculatus, Lotus pedunculatus, Lotus purshianus, Lotus angustissimus, Lotus tenuis, Lespediza stipulacea, Desmodium intortum, Desmodium uncinatum, Leucaena leococephala, Macrotyloma axillare, Stylosanthes gracilis, Trifolium dubium, Hordeum vulgare, Vitis vinifera, Calliandra spp, Arachis spp, Brachiaria spp., Codariocalyx spp, Gliricidia spp, Erythrina spp, Flemingia spp, Phyllodium spp., Tadehagi spp. or Dioclea spp., amongst others, where improved palatability or digestibility of said crop is desired.

The present invention is further described in the following non-limiting Examples. The examples herein are provided for the purposes of exemplification only and should not be taken as an intention to limit the subject invention.

### Example 1

### Materials and Methods

### Plant Material

Grapevine tissues of Vitis vinifera L. cv. Shiraz were collected from a commercial vineyard during the 2000-2001 season. Approximately 100 berries from at least 20 bunches were collected at weekly intervals throughout berry development from floral initiation until harvest, as described in Downey at al. (2003a). All samples were frozen in liquid nitrogen upon collection in the field and stored at −80° C. until analyzed.

Arabidopsis thaliana Columbia-0 (Col-0) and tt2 (SALK_{—}005260) seeds were provided by The Arabidopsis Biological Resource Center (Ohio, USA).

Preparation of cDNAs

Total grapevine RNA was isolated from the various plant tissues as described in Downey et al. (2003b). Arabidopsis RNA was isolated from leaves with RNeasy Kit (Qiagen) following the suppliers protocol. The quality of RNA was verified by demonstration of intact ribosomal bands following agarose gel electrophoresis in addition to the absorbance ratios (A260/280) of 1.8 to 2.0. For cDNA synthesis, four micrograms of grapevine or one microgram of Arabidopsis total RNA were reverse transcribed using oligo d(T)₁₈ and SuperScript™ III reverse transcriptase (Invitrogen Life Technologies) following the protocol of the supplier.

### Cloning of VvMYBPA1 and Plant Transformation

The ORF of VvMYBPA1 was inserted into the binary vector pART27 for expression of the gene in Arabidopsis. Therefore, the VvMYBPA10RF was amplified by PCR from V. vinifera L. cv. Shiraz cDNA (from RNA isolated ten weeks before veraison) using PfuTurbo® polymerase (Stratagene, USA) and the primers MybPAertF (5′-TGAGGTACCGAGAGAGATATGGGCAGAGCAC-′3; SEQ ID NO: 3) and MybPAartR (5′-TGAGGATCCTGATCTTTTGGTCTCTCTGCAA-′3; SEQ ID NO: 4). The generated PCR-fragment was purified, digested with BamHI and KpnI and cloned in the vector pART7 (Gleave, 1992), to give pART7MYBPA1 where VvMYBPA1 is under the control of the CaMV 35S promoter. The nucleotide sequence of the VvMYBPA1 ORE (Accession AM259485) in pART7MYBPA1 was determined by DNA sequencing. The expression cassette present in pART7MYBPA1 was isolated as NotI fragment, cloned into the NotI site of the binary vector pART27 (Gleave, 1992) and transferred into A. tumefaciens (AGLI) by electroporation. Arabidopsis tt2 (SALK005260) ecotype Columbia was transformed using the floral-dip method (Clough and Bent, 1998). T₁ transgenic plants were selected on one-half-strength Murashige and Skoog media containing 8 g/L agar and 35 mg/L kanamycin. Kanamycin-resistant T₁ seedlings were transferred to soil and grown at 20° C. in a growth chamber (Phoenix Biosystems, Adelaide, Australia) with a 16 h day length and a light intensity of 150 mmol m⁻² s⁻². Seeds of individual self-fertilized T2 lines were collected and single-copy insertion lines were selected based on a Mendelian segregation ratio.

### HPLC Analysis and DMACA Staining of Proanthocyanidins

Immature Arabidopsis siliques were finely ground and 80 mg were extracted in 400 μl 70% (v/v) acetone containing 0.1% (w/v) ascorbate for 18 h at room temperature on a rotating wheel in darkness. Samples were centrifuged and 300 μl aliquots of the supernatant were transferred to fresh tubes and vacuum dried at 35° C. for 60 min. The pellet was resuspended in 100 μl phloroglucinol buffer (0.25 g ascorbate, 1.25 g phloroglucinol, 215 μl conc. HCl, 25 ml methanol) and incubated at 50° C. for 20 min, then neutralized with 100 μl sodium acetate (200 mM, pH 7.5) for the analysis of PAs. Reverse-phase-HPLC was used for analysis of PAs as described by Downey et al. 2003a.

The presence of PAs in plant tissue was detected by staining the tissues with dimethylaminocinnamaldehyde (DMACA) solution (1% DMACA, 1% 6N HCl in methanol). Dried seeds were stained for 6-14 h and seedlings for 10-30 min. The tissues were then transferred to distilled water and blue staining of the tissue was visualized with a microscope and documented using a digital camera.

### Cloning of the Reporter and Effector Constructs for the Transient Promoter Assays

The Universal GenorneWalker™ Kit (Clontech, USA) was used to isolate promoter fragments of VvCHI, VvF3′5′H1, VvANR and VvLAR1. Four libraries of adaptor-ligated genomic fragments were constructed from V. vinifera (Shiraz) genomic DNA restricted by DraI, EcoRV, PvuII or StuI endonucleases and generated according to the GenomeWalker™ protocol. These genomic DNA-libraries served as templates for the promoter isolation. Outer and nested gene-specific primers were designed to the 5′ ends of the cDNA sequences of VvCHI (accession no. X75963), VvF3′5′H1 (accession no. AJ880356), VvANR (accession no. CAD91911) and VvLAR1 (accession no. AJ865336) and primary and secondary PCRs were performed with the outer adapter primer AP1 and the nested adapter primer AP2, respectively. Primer design and PCR conditions for genome walking were performed according to the manufacturer's instructions. The amplified promoter-fragments of the nested PCRs were cloned into pDrive (Qiagen, Germany) and sequenced. These DNA-sequences were then used to design specific primers for the amplification of the respective promoter from V. vinifera (Shiraz) genomic DNA using PfuTurbo® polymerase (Stratagene, OSA). The primers used for these PCR reactions contained restriction sites (in bold) for cloning the promoters into the luciferase reporter vector pLuc (Horstmann et al., 2004) as a BamHIH/XhoI fragment. Their DNA sequences were as follows:

ChIF
(5′-ATAGGATCCTGGAATTATGGAAGACAAATAGTCAA-′3; SEQ ID NO: 5),
CHIr
(5′-TTACTCGAGGATATGGCTGCAGAGAAACGA-′3; SEQ ID NO: 6),
ANRf
(5′-CGAGGATCCCATTCATAGTCAAATTACAAAAATCAA-′3; SEQ ID NO: 7),
ANRr
(5′-ATACTCGAGATATGCCCTCACTTCCAAATTC-′3; SEQ ID NO: 8),
F35Ht
(5′-CGAGGATCCCAAAAAGAGTTGGAAATACAACGA-′3; SEQ ID NO: 9),
F35Hr
(5′-ATACTCGAGTGACTATAGGATAGTGAAGGTGGCTAT-′3; SEQ ID NO: 10),
LARf
(5′-CGAGGATCCTCGGAATAATTTCATAGGGCTTT-′3; SEQ ID NO: 11)
and
LARr
(5′-ATACTCGAGTCTGATGATGCTTCTTCTCTACTACTC-′3; SEQ ID NO: 12).

A 1674 bp DNA-fragment of the VvUFGT promoter (accession no. AY955269) was amplified by PCR from the plasmid pART7UFGT:GFP (gift from Paul Boss, CSIRO, Australia) using Pfx polymerase (Invitrogen) with the primers UFGTpF (5′-ACGGGATCCTCATGCGTCCACCTATTATCAA-3′; SEQ ID NO: 13) and UFGTpR (5′-GTACTCGAGGGTTGGAATGGGGGATGTTA-3′; SEQ ID NO: 14). A 1533 bp DNA-fragment of the AtANR promoter was amplified with PfuTurbo® polymerase using the primers AtANRf (5′-CGAGGATCCCTGCGAAGACAATCCTCACT-′3; SEQ ID NO: 15) and AtANRr (5′-ATCTCGAGTTGAAATTACAGAGATAGAGATTTAGTTG-′3; SEQ ID NO: 16). A 2174 bp DNA-fragment of the VvLDOX promoter was amplified with PfuTurbo® polymerase using the primers LDOXf (5′-CGAGGATCCOTTTGCTTCCATCCCAATCTCACT-3′; SEQ ID NO: 17) and LDOXr (5′-TGTCTCGAGAAATATCACTGATCTACTTGTTTTCC-3′; SEQ ID NO: 18). These PCR-fragments were gel purified, digested with BamHI and XhoI and cloned between the respective sites of the vector pLuc. All described grapevine promoters were amplified from V. vinifera (Shiraz) genomic DNA.

For transient expression of TT2 and VvMYBA2 their ORFs were amplified from cDNA by PCR using PfuTurbo® polymerase and cloned into the vector pART7, which contains the CaMV 35S constitutive promoter. Therefore, TT2 was amplified using the primers TT2F (5′-AGGTCGACATGGGAAAGAGAGCAACTACTAGTG-3′; SEQ ID NO: 19) and TT2R (5′-TACTCGAGTCAACAAGTGAAGTCTCGGAGC-3′; SEQ ID NO: 20) from cDNA of Col-0 siliques. The PCR-fragment was digested with SalI and XhoI and ligated into pART7, digested with the same enzymes. The ORF of VvMYBA2 was amplified from grapevine post-veraison berry skin cDNA using the primers MybAF (5′-CGCCTCGAGCTCGATGGAGAGCTTAGGAGTTAG-3′; SEQ ID NO: 21) and MybAR (5′-CGCTCTAGATAAATCAGATCAAATGATTTACTT-3′; SEQ ID NO: 22). The PCR-fragment was digested with XhoI and XbaI and ligated into pART7, digested with the same enzymes. All described PCR fragments were subjected to DNA sequencing before analysis in the transient assay system.

### Transient Transfection Experiments and Dual-Luciferase Assay

A transient assay was developed using a cell suspension of a Chardonnay petiole callus culture, maintained on Grape Cormier (GC) medium. Cells in log-phase growth were gently filtered onto sterile Whatman discs and placed on GC media. Gold particles were coated with a mixture of DNA constructs (150 ng of the respective plasmid, giving a total plasmid concentration of 750 ng/shot) by the method described in Ramsay et al. (2003) and used to bombard Chardonnay cells at a helium pressure of 350 kPa within a vacuum of 75 kPa and a distance of 14 cm (Torregrosa et al., 2002). For the dual-luciferase assay, each bombardment contained a positive control of 3 ng of the Renilla luciferase plasmid pRluc (Horstmann et al., 2004). Cells were harvested 48 h after transfection and lysed by grinding on ice in 150 μl of Passive Lysis Buffer (PLB, Promega). After centrifugation of the lysates for 2 min at 500×g, measurement of the luciferase activities was performed with the dual-luciferase reporter assay system (Promega), by sequential addition of 25 μl LARII and Stop & Glo® to 10 μl of the lysate supernatant. Light emission was measured with TD-20/20 Luminometer (Turner Design) and the relative luciferase activity was calculated as the ratio between the firefly and the Renilla (control) luciferase activity. All transfections experiments were performed in triplicate and each set of promoter experiments was repeated with similar relative ratios to the respective control.

### Expression Analysis of VvMYBPA1

Transcript levels of VvMYBPA1 in grapevine were measured by Real Time PCR, using SYBER green method on a Rotor-Gene 2000 (version 4.2) real-time cycler (Corbett Research, Australia). Each PCR reaction (15 μl) contained: 266 nM primer (each), cDNA (diluted 1:60) and 1× ABsolute™ QPCR SYBR® Green ROX Mix (ABgene House, UK). The thermal cycling conditions were 95° C. for 15 min followed by 95° C. for 30 s, 58° C. for 25 s, and 72° C. for 25 s for 30 or 35 cycles, followed by a melt cycle from 50 to 96° C.

The EST clone TC46393 (TIGR database) was used to design the primers MYBPA1F (5′-AGATCAACTGGTTATGCTTGCT-3′; SEQ ID NO: 23) and MYBPA1R (5′-AACACAAATGTACATCGCACAC-3′; SEQ ID NO: 24) which were used to detect the transcript level of VvMYBPA1 in grapevine and amplified a 190 bp PCR-fragment from the 3′ untranslated region of the gene. With all cDNAs used the primer set gave a single PER product which was verified by determining the melt curves for the product at the end of each run, by analysis of the product using gel electrophoresis, and by comparing the DNA sequence of the PCR product with the gene sequence. The efficiency of the primers was tested in preliminary experiments with dilutions of the purified PCR product and maintained an r² value ≧0.98. The expression of genes was normalized to VvUbiquitin1 (TC32075, TIGR database), which transcripts were detected by amplifying a 182 bp product with the primers VvUbiquitin Forward (5′-GTGGTATTATTGAGCCATCCTT-3′; SEQ ID NO: 25) and VvUbiquitin Reverse (5′-AACCTCCAATCCAGTCATCTAC-3′; SEQ ID NO: 26). All samples were measured in triplicate. The difference between the cycle threshold (Ct) of the target gene and the Ct of Ubiquitin, ΔCt=Ct_{Target}−Ct_{Ubiquitin}, was used to obtain the normalized expression of target genes, which corresponds to 2^{-ΔCt}. The Rotor Gene 2000 software (Corbett Research, UK) and the Q-Gene software (Muller et al., 2002) were used to calculate the mean normalized expression of the genes.

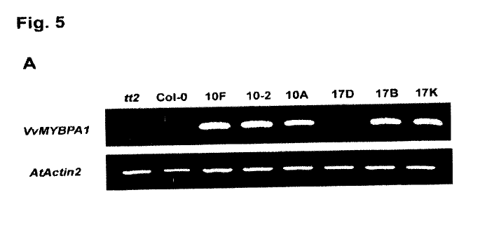

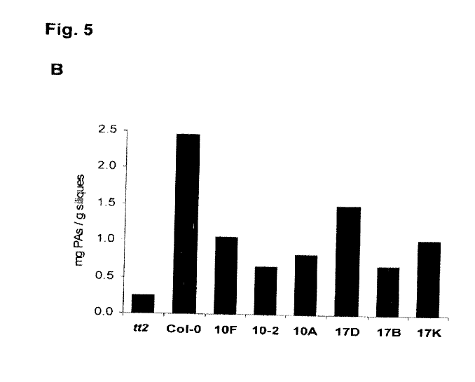

For detection of the VvMYBPA1 transcript in Arabidopsis (FIG. 5), PCR reactions were performed as described above and analyzed on a 1.5% agarose gel containing ethidium bromide. The primers MYBf (5′-CAACTGACAACTCTCTGGACAA-3′; SEQ ID NO: 27) and MYBr (5′-GATCTTTTGGTCTCTCTCCAAC-3′; SEQ ID NO: 28) were used to amplify a 116 bp PCR from the 3′ translated region of the gene. To determine whether the similar amounts of cDNA were applied to all samples, a 268 bp PCR fragment from the Arabidopsis Actin2 gene (accession number NM_{—}112764) was amplified with the primers ACT2F (5′-ATTCAGATGCCCAGAAGTCTTGTTCC-3′; SEQ ID NO: 29) and ACT2R (5′-ACCACCGATCCAGACACTGTACTTCC-3′; SEQ ID NO: 30).

### B. Results

### The Grapevine Gene VvMYBPA1 Encodes a MYB Transcription Factor

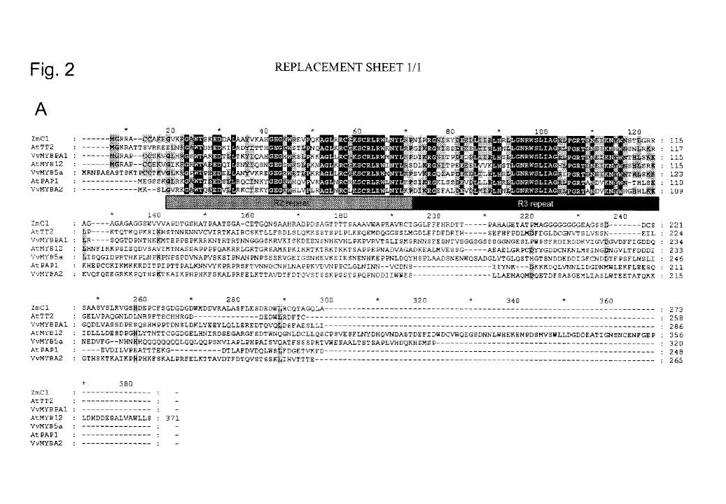

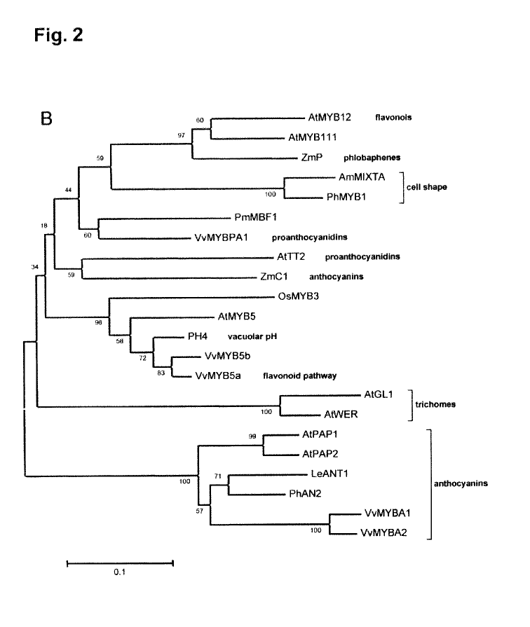

The Tentative Consensus (TC) sequence TC46393 was identified by searching the grape gene index of the TIGR EST-database (Quackenbush at al., 2000, http://www.tigr.org/tdb/tgi/) for MYB transcription factors expressed during early flower and berry development when PAs are accumulating. The 861-bp open reading frame (ORE) of TC46393 was amplified by PCR from cDNA isolated from Shiraz flowers sampled one week after flowering. The isolated ORE was named VvMYBPA1 (Accession AM259485) and encoded a protein of 286 amino acid residues with a predicted mass of 32.2 kD and a calculated pI of 9.47. Analyses of the deduced amino acid sequence revealed that VvMYBPA1 contains an N-terminal R2R3 repeat that corresponds to the DNA binding domain of plant MYB-type proteins (FIG. 2A). Similar to the over 100 members of the MYB protein family in Arabidopsis, the R2R3 repeat region of VvMYBPA1 is highly conserved and contains the motif [D/E]Lx₂[R/K]x₃Lx₆Lx₃R for interaction with basic helix-loop-helix (bHLH) proteins, whereas the C-terminal region shows little homology to other MYBs (FIG. 2A) (Stracke et al., 2001). Phylogenetic analysis revealed the similarity of VvMYBPA1 to other plant MYB proteins (FIG. 2B). The R2R3 DNA binding domain of VvMYBPA1 is most closely related to PmMBF1 (AAA82943) of Picea mariana with al % identical amino acid residues. The PmMBF1 protein has not yet been functionally characterized. The similarity between the MYB domain of VvMYBPA1 and AtTT2 (FIG. 2A), which was shown to regulate PA synthesis in the seed coat of Arabidopsis (Nesi et al., 2001), is also obvious with 72% amino acid identity. Besides VvMYBPA1 and TT2, the maize MYB factor C1 was also shown to activate the Arabidopsis ANR promoter, which is the branch point gene leading to PA synthesis (Baudry et al., 2004). FIG. 2A shows the comparison of these MYB proteins with the anthocyanin specific factors VvMYBA2 and AtPAP1 as well as with AtMYB12, a transcription factor controlling flavonol synthesis (Mehrtens et al., 2005). The alignment shows that there are no conserved amino acids in the sequences of VvMYBPA1, TT2 and C1 which are not also present in the other MYB factors (FIG. 2A). Sequence similarity between MYB proteins is generally restricted to the R2R3 domain, but some MYB factors share conserved motifs in their C-terminal domains that may indicate similarities in function (Stracke et al., 2001). However, the C-terminal sequences of VvMYBPA1 (amino acids 116-286) and any other plant MYB factors showed no significant homology and none of the conserved motifs identified by Strake et al, (2001) was present. Also the motif Vx₂IRTKA[I/L]RC[S/N] conserved between TT2 and OsMYB3 (Nesi et al., 2001) was not found in the sequence of VvMYBPA1 (FIG. 2A).

Taken together, the VvMYBPA1 protein sequence shows the typical features of a plant MYB transcription factor. However, conserved amino acid homologies between the PA regulators TT2 and VvMYBPA1, which could be used to identify PA specific MYB regulators from other plant species, were not detected.

Expression of VvMYBPA1 During Grape Berry Development Correlates with PA Accumulation

To confirm that VvMYBPA1 is expressed when PAs are accumulating in grape berries, transcript levels of VvMYBPA1 throughout grape berry development (V. vinifera L. cv Shiraz) were investigated during the season 2000-2001 by real-time PCR. VvUbiquitin1 (BN000705) was chosen for normalization of gene expression because it was found to be relatively constant throughout grape berry development (Downey et al., 2003b; Bogs et al., 2005).

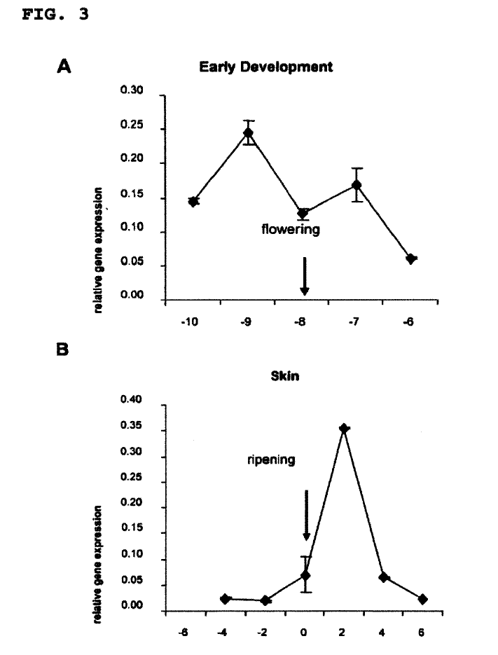

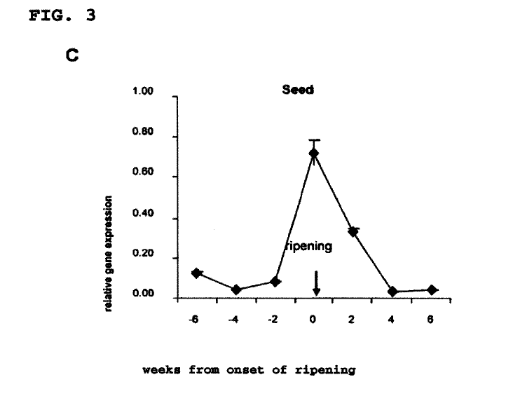

FIG. 3 shows VvMYBPA1 is expressed in flowers and grapes early in berry development from ten to six weeks before onset of ripening. This early expression of VvMYBPA1 in developing flowers and grape berries correlates with the accumulation of PAs and the expression of the structural genes VvLDOX (leucoanthocyanidin dioxygenase), VvANR (anthocyanidin reductase) and VvLAR1 (leucoanthocyanidin reductase) which are involved in PA synthesis in grapevine (Bogs et al., 2005).

In grape berry skins, transcript levels of VvMYBPA1 were relatively low before veraison, which is the onset of ripening, increased to a maximum two weeks after veraison and then declined to a low level (FIG. 3). The concentration of PAs in skins increased from five weeks before veraison, reaching a maximum around the time ripening commenced and then declined during ripening (Bogs at al., 2005). In seeds, VvMYBPA1 is expressed before veraison (FIG. 3) when PAs start to accumulate. The expression pattern of VvMYBPA1 in seeds (FIG. 3) correlates with PA synthesis and the expression of VvLAR2 which continued in the seed up until four weeks after veraison with a maximum at veraison (Kennedy at al., 2000; Bogs at al., 2005).

### Promoter Isolation and Analysis of Grapevine Flavonoid Path a Genes

To determine which genes of the flavonoid pathway are controlled by VvMYBPA1, the promoter regions of the genes VvF3′5′H1 (1136 bp, accession AM259482), VvCHI (935 bp, accession AM259483), VvANR (1034 bp, accession AM259484) and VvLAR1 (1342 bp, accession AM259481) were isolated by genome walking (see Material and Methods). These promoter regions were analyzed using the PLACE (plant DNA cis-elements) database (Higo et al., 1999; http://www.dna.affrc.go.jp/htdocs/PLACE/signalscan.html) and contained the consensus sequences of the core DNA binding sites of MYB (CNGTTR, PLACE accession S000176) and NYC-type (CANNTG, PLACE accession 5000407) transcription factors. The core MYB site CNGTTR is recognized by the plant transcription factor MYB.Ph3 from petunia (Solano et al., 1995), which is involved in regulation of flavonoid biosynthesis and is present in a 86 bp promoter region of the Arabidopsis AtANR promoter necessary for expression in PA-accumulating cells (Debeaujon et al., 2003). Additionally, the promoter regions of VvLDOX (2174 bp of accession AF290432), VvUFGT (1674 bp of accession AY955269) and AtANR (also called AtBAN, 1533 bp of accession AT1G61720), which also contain the core DNA binding sites for MYB- and NYC-factors (Gollop et al., 2001; Kobayashi et al., 2001) were cloned. There are different target recognition sites for different groups of MYB proteins (Jin and Martin, 1999) and in addition to the core MYB DNA binding site, all promoters contained putative cis-acting regulatory elements for different MYB proteins (data not shown).

### VvMYBPA1 Activates Promoters of the Flavonoid Pathway Genes Involved in PA Synthesis

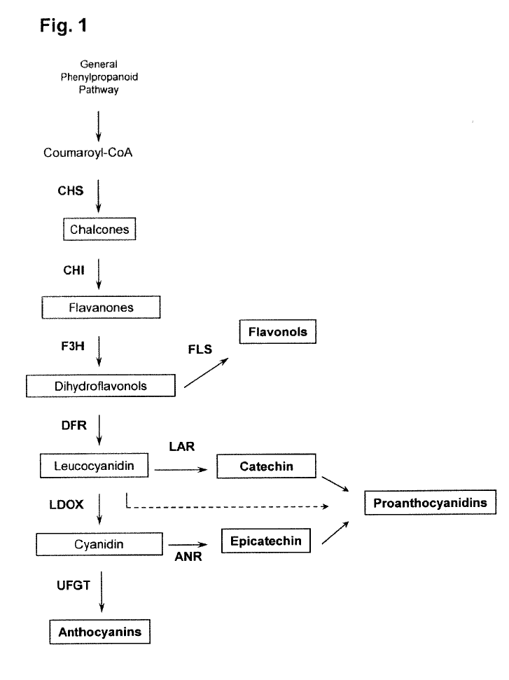

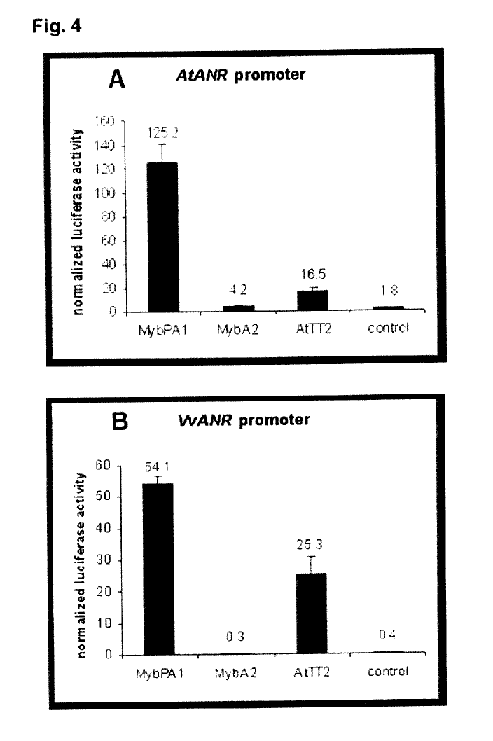

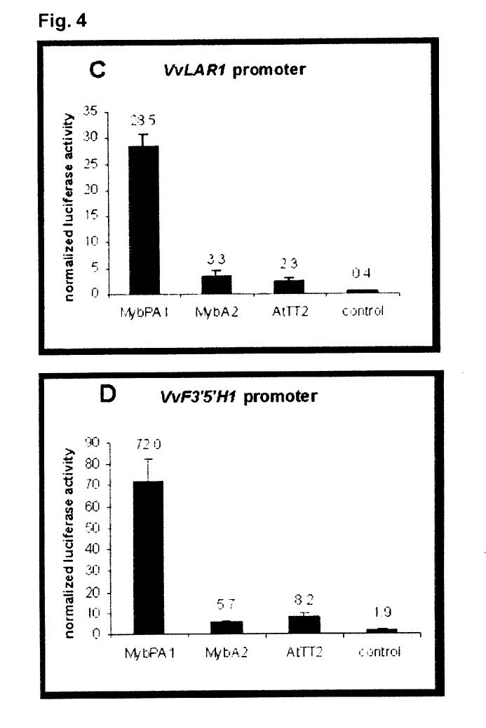

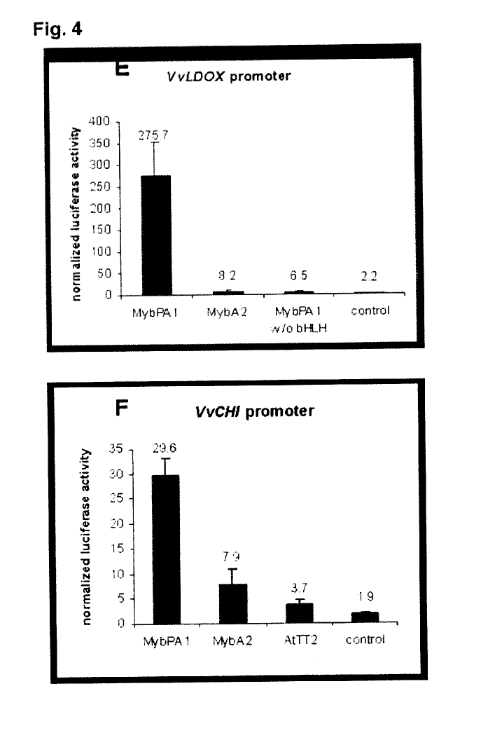

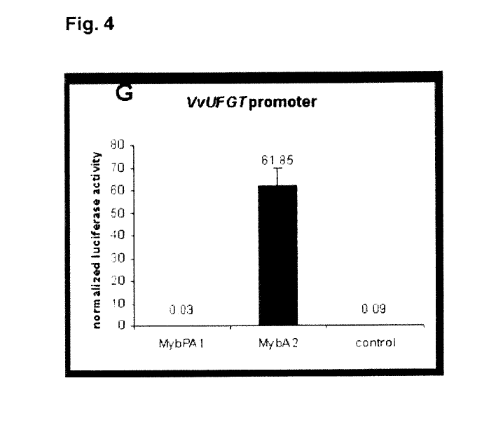

To investigate which structural, genes of the flavonoid pathway are activated by VvMYBPA1, a transient expression method was established using grape cell culture and the dual-luciferase assay system. In this system, the cotransfection at effectors (transcription factors) and dual-luciferase reporter plasmids allows quantification of promoter activity by measuring firefly luciferase activity (promoter of interest cloned into pLuc), which is normalized by measuring Renilla reniformis luciferase activity (pRluc) (Horstmann at al., 2004). Therefore, VvMYBPA1 was ligated to pART7 to be expressed under the control of the 35S promoter of Cauliflower mosaic virus The promoters controlling VvCHI (chalcone isomerase), VvF3′5′H1 (flavonoid 3′,5′-hydroxylase), VvLDOX, VvANR, AtANR, VvLAR1, and VvUFGT were ligated to pLuc to control the expression of the firefly luciferase reporter gene. The VvCHI, VvF3′5′H1 and VvLDOX promoters were chosen as examples for flavonoid general pathway genes involved in synthesis of flavonols, anthocyanins and PAs (FIG. 1). In contrast, VvUFGT is specifically involved in anthocyanin synthesis, whereas VvANR and VvLAR1 encode the branch point enzymes leading to the synthesis of PAs (FIG. 1). Except for VvLAR1 and VvF3′5′H1, these genes are present as a single copy in the grapevine genome (Sparvoli et al., 1994; Bogs et al., 2005). The grapevine promoters and the Arabidopsis AtANR (also called BANYULS) promoter were then tested as potential targets for VvMYBPA1 (FIG. 4). As controls, the promoters were tested also with the MYB transcription factor VvMYBA2 which activates VvUFGT controlling anthocyanin synthesis in grapes (Boss et al., 1996; Kobayashi et al., 2002) and TT2 from Arabidopsis which was shown to control AtANR expression and PA synthesis in Arabidopsis seed coat (Nesi et al., 2001). Similar results as for VvMYBA2 were obtained with its isoform VvMYBA1 when transfected with the VvUFGT or VvANR promoter and EGL3 in the transient expression system (data not shown).

VvMYBPA1 strongly activated the promoters of rice genes VvANR (˜135 fold), AtANR (˜70 fold) and VvLAR1 (˜72 told) showing its ability to induce the PA specific branch point genes of Arabidopsis and grapevine (FIG. 4A-C). VvMYBPA1 also induced the promoters of the general flavonoid pathway genes VvCHI (˜16 fold), (˜38 fold) and VvLDOX (˜125 fold) suggesting it can activate the whole pathway leading to PA synthesis (FIG. 4D-F). The anthocyanin specific promoter of VvUFGT was not affected by VvMYBPA1, whereas VvMYBA2 strongly activated (˜600 fold) this promoter (FIG. 4G). In comparison to VvMYBPA1, the activation of the VvF3′5′H1, VvLDOX, VvCHI, VvANR, VvLAR1 and the AtANR promoter by VvMYBA2 was absent or relatively low (FIG. 4). Similar to VvMYBPA1, the Arabidopsis PA regulator TT2 activated the ANR genes of Arabidopsis and grapevine (FIGS. 4A and B). In contrast to VvMYBPA1, TT2 was not able to induce the promoters of VvCHI and VvLAR substantially (FIGS. 4C and F). These results suggest VvMYBPA1 is a specific regulator of PA synthesis, potentially regulating the entire general flavonoid pathway and the branch point genes ANR and LAR leading to PA formation.

Similar to other MYB transcription factors, VvMYBPA1 requires a bHLH protein for promoter activation (FIG. 4E; VvMYBPA1/w/o). Therefore, all standard transfections included a construct expressing EGL3 which encodes a bHLH protein involved in flavonoid pathway regulation in Arabidopsis (Ramsay et al., 2003). FIG. 4 also shows that using VvMYBPA1 without a bHLH protein in these transfection experiments can induce the VvLDOX promoter up to three fold compared to the control without the MYB factor and similar results (3-5 fold inductions) were obtained for other promoters and MYB factors (data not shown). However, in comparison to the 125 fold induction of the VvLDOX promoter by VvMYBPA1 and EGL3 a three fold induction was considered as insubstantial and possibly provoked by the large amounts of MYB factor and promoter DNA in the transfection assay

In Arabidopsis, the endogenous bHLH protein TTS has been shown to interact with TT2 and to be required for PA accumulation in the seed coat (Nesi et al., 2000). Therefore, the ability of TT2 and VvMYBPA1 to interact with TT8 and to activate the ANR promoters of Arabidopsis and grapevine was also tested. It was found that the ANR promoter activities were not substantially altered when EGL3 was exchanged with TT8 in the transient assays (data not shown). This redundancy of EGL3 and TT8 was also found for their ability to interact with the MYB factors TT2, PAP1 or PAP2 and to activate the AtDFR promoter of Arabidopsis (Zimmermann et al., 2004).

VvMYBPA1 Complements the Arabidopsis tt2 Mutant PA-Deficient Phenotype

The MYB transcription factor TT2 was shown to regulate PA synthesis in the seed coat of Arabidopsis and the seeds of tt2 mutants appear yellow due to the lack of PAs (Nesi et al., 2001). To confirm the function of VvMYBPA1 as a regulator of PA synthesis, VvMYBPA1 under the control of the CaMV 35S promoter was introduced into the tt2 mutant. The ORF of VvMYBPA1 was amplified from cDNA by PCR and inserted into the binary vector pART27 to give pART27MYBPA1, which contains kanamycin resistance for selection in planta. This construct was used to transform homozygous tt2 plants by A. tumefaciens mediated transformation.

About 80% of the transgenic kanamycin-resistent T₁ seedlings showed growth abnormalities with bleaching and necrosis of the first leaves during their development. These plants showed a dwarf phenotype and died 1-3 weeks after transferring them to soil. Dimethylaminocinnamaldehyde (DMACA) is a useful reagent for detection of molecules of the PA pathway because it reacts with PA monomers as well as their polymers to form a blue chromophore but does not react with anthocyanidin derivatives (Nagel and Glories 1991). When these seedlings were stained with DMACA, accumulation of PAs was observed in cells of cotyledons, hypocotyls and its apical meristem, roots, basal cells of trichomes and trichomes indicated by their blue staining. Control plants (tt2) stained with DMACA did not show any blue staining indicating their inability to accumulate significant amounts of PAs.

About 20% of the transformants did survive and were grown on to produce seed. Nine kanamycin-resistant tt2 plants transformed with pART27MYBPA1 showed wild-type phenotype and developed brown seeds which stained blue for accumulation of PAs when stained with DMACA. This demonstrated that ectopic expression of VvMYBPA1 can complement the tt2 mutant seed phenotype. From the independent tt2 35S::MYBPA1 lines 10 and 17, T₂ plants were generated and analyzed for expression of VvMYBPA1 and PA accumulation. Expression of VvMYBPA1 by the tt2 35S::MYBPA1 lines 10F, 10-2, 10A, 17D, 17B and 17K was confirmed by RT-PCR (FIG. 5A). Blue PA staining was observed in their hypocotyls, roots, seeds, bases of the rosette leaves and stipules of leaves (data not shown) after these plants were treated with DMACA, whereas Col-0 wild-type plants accumulated PAs exclusively in the developing seeds. HPLC analysis of the developing siliques revealed that the PA levels of tt2 plants complemented with VvMYBPA1 were 3-8 fold higher than in the tt2 background and reached about half of the PA concentration we detected in the Col-0 wild-type siliquas (FIG. 5B).

### C. Discussion

### VvMYBPA1 Encodes a MYB-Type Transcriptional Regulator

Proanthocyanidins (PAs) are important quality components of many fruits, but little is known about regulation of PA synthesis in fruit. The transcription factor TT2 from Arabidopsis was the first MYB protein shown to determine specifically PA accumulation (Nesi et al., 2001) and until now no functionally homologous MYB factors from other plant species have been described. In this study, it is shown that the grapevine MYB regulator VvMYBPA1 can complement mutations in tt2 and evidence is presented that VvMYBPA1 specifically regulates PA synthesis during grape berry development. The protein sequence of VvMYBPA1 shows homology to the R2R3 domain of various MYB transcription factors (FIG. 2). However, it was not possible to find any of the conserved motifs described by Stracke et al, 2001 in its C-terminal domain that may indicate similarities in function (FIG. 2). VvMYBPA1 does not display significantly more similarity to TT2 or C1 from maize, which have been shown to activate the ANR (BAN) promoter (Nesi et al., 2001), than to any other functionally unrelated MYB regulator (FIG. 2). Therefore it was not possible to identify conserved amino acid homologies or motifs, which could be used to identify PA specific MYB regulators from other plant species. However, the transient expression assays and complementation experiments showed that VvMYBPA1 can replace TT2 suggesting that VvMYBPA1 and TT2 are orthologous MYB factors. Similar findings were described for AN2 from petunia and C1 from maize, where functional homology of the proteins in regulating anthocyanin synthesis was no': reflected in specific amino acid similarities (Quattrocchio et al., 1998 and 1999).

Expression of VvMYBPA1 Correlates with PA Synthesis

The functional role of VvMYBPA1 in the regulation of PA synthesis in grape berries is supported by its gene expression pattern during grape berry development (FIG. 3). Development of the grape berry occurs in two successive growth phases and the synthesis of flavonols, anthocyanins and PAs and the expression of flavonoid pathway genes is temporally separated during berry development. The first phase, from around flowering until the onset of ripening (veraison), coincides with flavonol and PA synthesis and the second phase, starting with the onset of ripening of the berry, coincides with anthocyanin biosynthesis (Robinson and Davies, 2000; Downey et al., 2003a and b; Bogs et al., 2005). The biosynthesis of PAs, anthocyanins and flavonols share common steps in the flavonoid pathway, whereas the activities of branch point enzymes specific for PAs, anthocyanins or flavonols lead exclusively to the synthesis of the respective flavonoid (FIG. 1). Therefore, regulation of this pathway must occur to coordinate synthesis of different flavonoids during grape berry development. Most of the regulation of flavonoid synthesis occurs via coordinated transcriptional control of the structural genes by the interaction of DNA-binding MYB transcription factors and MYC-like basic helix-loop-helix (bHLH) proteins (Mol et al., 1988). It has been demonstrated that VvMYBPA1 is a MYB transcription factor which is expressed during flower and early berry development and in seeds before ripening (FIG. 3). This expression pattern correlates with PA accumulation and expression of the PA branch point genes VvLAR1, VvLAR2 and VvANR (Bogs et al., 2005). PA synthesis appears to continue in the seed up until 2-4 weeks after veraison (Kennedy at al., 2000), which coincides with the expression pattern of VvLAR2 (Bogs at al., 2005) and VvMYBPA1 in seeds (FIG. 3). Both, VvLAR2 and VvMYBPA1 expression reached their maximum in seeds at veraison and this corresponds to the peak of PA monomer accumulation around vera son (Bogs et al., 2005). In grape skins, PA accumulation appeared to be complete by veraison and maximum transcript levels of VvANR and VvLAR2 were already detected four weeks before veraison (Bogs et al., 2005). The transcript level of VvMYBPA1 in skins was relatively low before veraison, increased to a maximum two weeks after veraison and then declined to a low level (FIG. 3). It is unclear if VvMYBPA1 expression before veraison is sufficient to induce PA synthesis in skins or another regulator activates PA synthesis in this tissue. However, a maximum of VvMYBPA1 expression in skins could take place earlier than four weeks before veraison, where no expression data has been obtained because it was not possible to separate seeds and skins from very small berries. This would explain VvMYBPA1 expression in skins before veraison, but the reason for its relatively high transcript level two weeks after veraison remains unclear. Taken together, the expression pattern of VvMYBPA1 suggests that the encoded protein is involved in regulation of PA biosynthesis in grapevine at least during early fruit development and in seeds.

### VvMYBPA1 Activates the Promoters of General Flavonoid Pathway and PA Branch Point Genes

Another important aspect of this study supporting the evidence that VvMYBPA1 specifically regulates PA biosynthesis was the ability of the transcription factor to exclusively activate promoters of flavonoid pathway genes involved in PA synthesis (FIG. 4). Anthocyanin and PA biosynthesis share the general flavonoid pathway enzymes until LDOX catalyses the synthesis of anthocyanidins (Abrahams et al., 2002), which are substrates for both anthocyanin synthesis via UFGT and PA synthesis catalyzed by ANR and LAR (FIG. 1). Therefore, UFGT and ANR/LAR are branch point enzymes leading to anthocyanin or PA accumulation and represent a possible control point between the two flavonoid branches. The results obtained with the transient promoter assay revealed the ability of VvMYBPA1 to activate the PA specific branch point genes VvANR and VvLAR showing its capacity to control PA synthesis in grapevine. Further, VvMYBPA1 was not able to induce the VvUFGT promoter suggesting it specifically regulates PA biosynthesis. In addition, it has been determined that VvMYBA2, which was shown to induce anthocyanin accumulation in grapes (Kobayashi et al., 2002), activates the VvUFGT promoter but not the VvANR or VvLAR promoter showing it specifically controls anthocyanin synthesis. These results suggest that in grapes the transcription factors VvMYBA2/VvMYBA1 and VvMYBPA1 control whether anthocyanins or PAs are synthesised by regulating expression of VvUFGT and VvANR/VvLAR, respectively. However, it cannot be excluded that there are additional MYB transcription factors involved in regulation of anthocyanin and/or PA synthesis in grapevine.

Similar to grapevine, the Arabidopsis MYB transcription factor TT2 controls PA synthesis and PAP1/PAP2 control anthocyanin biosynthesis. However, the Arabidopsis flavonoid MYB factors seem to control different steps in the flavonoid pathway than the functional homolcgues from grapevine. In previous studies it was shown that TT2 controls expression of the flavonoid “late” biosynthetic genes (LBGs) including DFR, LDOX, AtANR (BAN) and TT12, whereas transcript levels of the flavonoid “early” biosynthetic genes (EEGs) like CHS, CHI, F3′H or F3H were not affected by TT2 (Nesi at al., 2001). In contrast, the quantitative analysis of VvCHI, VvF3′5′H1, VvLDOX, VvANR, and VvLAR1 promoter activities revealed that VvMYBPA1 is able to induce promoters of EBGs and LBGs (FIG. 4). This could be due to different binding sites in the promoters of grapevine and Arabidopsis, differences of the MYB factors which change their ability to bind the promoters and/or other unidentified factors which are present in the grapevine cell cultures. Comparing binding activities of TT2 and VvMYBPA1 to homologous flavonoid promoters from Arabidopsis and grapevine could answer at least some of these questions. The transient expression experiments also showed that the activation of the AtANR promoter and VvANR promoter by VvMYBPA1 was eight fold and two fold higher than the respective activation by TT2 (FIGS. 4A and B).

### VvMYBPA1 Complements the tt2 Seed Phenotype and can Induce Ectopic PA Accumulation in Arabidopsis

By complementation of the PA deficient seed phenotype of the tt2 mutant it has been demonstrated that VvMYBPA1 is a grapevine functional orthologue of the Arabidopsis PA regulator TT2. Although TT2 controls PA synthesis in the Arabidopsis seed coat and its ectopic expression was shown to induce expression of the AtANR promoter, Arabidopsis 70S::TT2 (two copies of 35S promoter) plants failed to accumulate PAs in any tissue other than seeds (Nesi et al., 2001). In contrast, it has been shown that, in addition to seeds, Arabidopsis 35S::MYBPA1 plants accumulate PAs in their cotyledons, hypocotyls, roots, trichomes and basis of rosette leaves. A similar organ and cell specific pattern of PA accumulation was observed in Arabidopsis plants simultaneously over-expressing TT2 and PAP1, which reflected the ANR promoter activity in Arabidopsis (Sharma et al., 2005). The promoter studies (FIG. 4) and analysis of the 35S::MYBPA1 Arabidopsis plants suggest that in grapevine VvMYBPA1 regulates the whole flavonoid pathway branch leading to PA synthesis (flavonoid EBGs and LBGs), whereas in Arabidopsis TT2 controls only the flavonoid LBGs (Nesi et al., 2001). Therefore, co-expression of TT2 and PAP1 was needed for induction of the flavonoid EBGs and LBGs and ectopic formation of PAs in Arabidopsis (Sharma et al., 2005). The majority of the 35S::MYBPA1 transgenic seedlings developed growth abnormalities and accumulated relatively high levels of PAs in their roots, hypercotyl and the apical meristem, which could have lead to the death of these plants before they fully developed their first leaves. Presumably, these plants expressed much higher levels of VvMYBPA1 transcript than the lines which grew past this stages (e.g. line 17 and 10). Similar toxic effects for Arabidopsis were observed by constitutive co-expression of TT2, PAP1 and Lc which lead to PA formation in roots and leaves and death of the plants (Sharma et al., 2005). As Arabidopsis wild-type plants accumulate PAs only in the seed coat, transgenic plants ectopically synthesizing PAs are maybe not able to compartmentalize them into specific cell types or vacuoles and they become toxic for these plants.

It would be interesting if ectopic expression of VvMYBPA1 in plants like alfalfa and clover can induce PA formation in leaves or other tissue, because it is of great interest to engineer PAs in forage crops to reduce the risk of pasture bloat for ruminants (Dixon et al., 2005). Recently, Xie et al, (2006) have shown that expression of ANR in Meticago sativa or co-expression of ANR and PAP1 in tobacco can induce ectopic PA accumulation.

Unlike Arabidopsis, grapevine synthesises PAs of different polymer length and composition in leaves, flowers, and in the skin and seeds of the developing fruit (Kennedy et al., 2001; Bogs et al., 2005). There is considerable interest in grape PAs because of their importance for the flavor of wine and their antionidant capacity promoting health-benefits in a number of model cell and animal systems (Bagchi et al., 2000). The data suggest that VvMYBPA1 regulates PA formation in grapes and alteration of VvMYBPA1 expression may have the potential to manipulate the amount and composition of PAs in grapevine and other plants.

### Example 2

### Functional Analysis of Grapevine VvMYBPA1 Gene in Tobacco

To test the function of VvMYBPA1, it was introduced into tobacco under the control of the 35S CaMV promoter.

Methods: Transformation of Tobacco with VvMYBPA1 and Analysis of Flavonoids.

The full length VvMYBPA1 cDNA was cloned into the vector pART7 (Gleave, 1992) and transformed into tobacco via the pART27 vector under the control of the CaMV 35S promoter. Leaf disks of Nicotiana tabacum (cv.Samsun NN) were used for standard Agrobacterium-mediated transformation according to Horsch et al, 1985.

Proanthocyanidins (PAs) in transgenic tobacco petals were determined by extracting the petal tissue into 70% acetone with 0.1% ascorbate and quantitating PAs with the dimethylaminocinnamaldehyde (DMACA) reagent described by Nagel and Glories (1991) using catechin (Sigma) as a standard. Anthocyanins and flavonols in transgenic tobacco petal tissues were extracted into methanol/HC as described in Downey at al. (2004). Anthocyanin and flavonol content and composition were determined by reverse-phase HPLC using a HP1100 system (Agilent), with a Wakosil analytical column (150 mm×4.6 mm; 3 pm packing; SGE, Australia). The HPLC separation utilised a binary solvent gradient where Solvent A was 10% formic acid (v/v with water) and Solvent B was methanol. The gradient conditions were: zero minutes, 17% Solvent B; 15 minutes, 35% Solvent B; 40 minutes, 37% Solvent B; 42 minutes, 100% Solvent B 44 minutes, 100% Solvent B; 45 minutes, 17% Solvent B; 46 minutes, 17% Solvent B. The column was maintained at 40° C. and the flow rate was 1.0 mL/minute. Anthocyanins and flavonols were expressed as malvidin-3-glucoside and quercetin-3-glucoside equivalents respectively based on commercial standards (Extrasynthese, France).

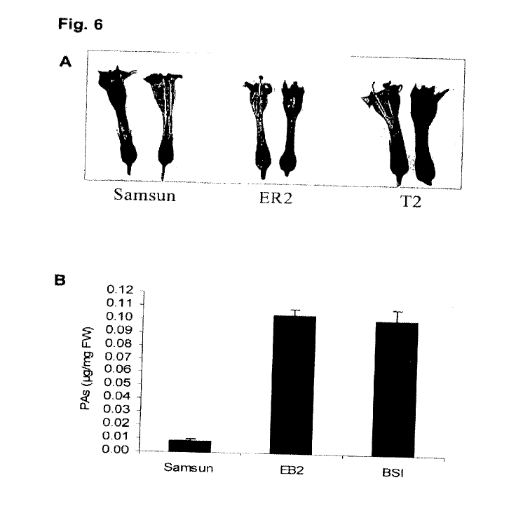

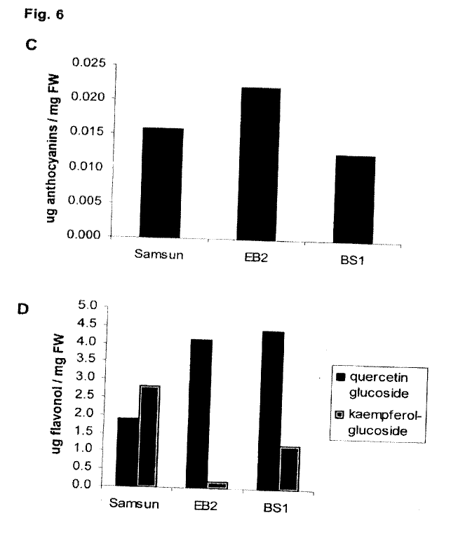

Several transgenic lines were generated and the petals of the flowers and the anthers showed proanthocyanidins (PAs) accumulation after staining with dimethylaminocinnamaldehyde (DMACA) indicated by the blue coloration (FIG. 6A). The petals of transgenic plants EB2 and T2 showed blue staining after DMACA treatment, while the petals of untransformed SamSun plants did not. Other tissue like leaves, stems or roots showed no signification blue staining after DMACA treatment. The level of PAs in the petals of transgenic lines EB2 and BS1, quantitated with DMACA, was more than 10-fold higher than in the petals of the untransformed Samsun plants (FIG. 6B). HPLC analysis of anthocyanins and flavonols in the petals of transgenic plants EB2 and BS1 and untransformed Samsun plants showed that the total amount of these flavonoids was not significantly altered (FIGS. 6C and D). However, whereas similar amounts of the flavonols kaempferol-glucoside and quercetin-glucoside were detected in untransformed Samsun plants, the transformed plants synthesized 3-10 fold more quercetin-3-glucoside than kaempferol-3-glucoside (FIG. 6D).

### Example 3

### Transformation of White Clover with VvMYBPA1 Gene

Transformation experiments to introduce the VvMYBPA1 gene are carried out with the white clover cultivars ‘Haifa’ and ‘Waverley’, using a binary transformation plasmid comprising the protein coding region (ORF) of VvMYBPA1 inserted into the binary vector pART27 (Gleave, 1992) where it is under the control of the CaMV 35S promoter. The plant selectable marker is nptII flanked by nos 5′ and nos 3′ sequences (described by An et al., 1985). The binary plasmid is transferred into A. tumefaciens (AGL1) by electroporation. Agrobacterium tumefaciens strain AGL1 carries a disarmed Ti plasmid (Lazo et al., 1991).

### White Clover Transformation

The method of Larkin et al., (1996) is followed. White clover seed is surface sterilised by soaking in 70% (v/v) ethanol for 3 min, 30% (v/v) bleach solution (final 1.5% (w/v) available chlorine) for 40 min, 70% ethanol again for 3 min followed by 6 washes in sterile distilled water over 1 h. These seeds are allowed to imbibe overnight in the dark at 15° C. for 17 h. The seeds are dissected under a binocular microscope to separate the imbibed cotyledons. These cotyledons are cut from the hypocotyl and epicotyl such that a small portion of the stalk is included, but not the cotyledonary node joining it to the hypocotyl. The cotyledons are collected into MG broth (Garfinkle, 1980) in a Petri dish. The Agrobacterium tumefaciens culture is grown at 27° C. for 20-24 h in MG broth up to a cell density of 3−5×10⁹ cells/ml. The cotyledons are transferred to the agrobacterial suspension in a shallow layer and gently agitated for 40 min. Following this incubation the cotyledons are transferred onto sterile filter paper to absorb excess suspension. The cotyledons and adhering agrobacteria are co-cultivated at 24° C. in the light for 3 days on agar medium B5PB. This medium contains the basal salts, vitamins and sugars of B5 (Gamborg et al., 1968) with 12 nM picloram, 2.2 μM BAP and 0.7% agar. After 3 days the cotyledons are collected, washed several times with sterile water, blotted with filter paper and transferred to B5PB with 300 μg/ml of antibiotic mix Timentin™ (Beecham Res. Labs.; a 30:1 w/w mixture of sodium ticarcillin and potassium clavulanate) to inhibit the further growth of the bacteria, and with 25 μg/ml of kanamycin to select for transformed plant cells.

After 3 weeks, cotyledons with green shoot initials are transferred to the same medium with Timentin and kanamycin for another 3 weeks. Green shoots are then transferred to the rooting medium, RIB. RIB contains the basal salts and organics of L2 (Phillips and Collins, 1984) with 1.2 μM IBA. If the shoots are already large the RIB is without PPT, but if the shoots are still small, the rooting medium contains 25 μg/ml kanamycin to safeguard against non-transgenic escape. Although there are often multiple shoots, only one green plantlet is chosen from each cotyledon to ensure all regenerants are from independent transfcrmation events. After forming roots within 2 or 3 weeks, plantlets are transferred to soil, but only after confirmation of their transformed status.

Alternatively, the method of Voisey et al., (1994) using direct shoot organogenesis is used for Agrobacterium mediated transformation of the white clover.

### REFERENCES

Abrahams S, Tanner G J, Larkin P J, Ashton A R (2002) Identification and biochemical, characterization of mutants in the proanthocyanidin pathway in Arabidopsis. Plant Physiol 130: 561-576 ,
An, G., et al. (1985), New cloning cehicles for transformation of higher plants. EMBO J., 4, 277-84.
Bagchi D, Bagchi M, Stohs S J, Das D R, Ray S D, Kuszynski C A,
Joshi S S, Pruess H G (2000) Free radicals and grape seed proanthocyanidin extract: importance in human health and disease prevention. Toxicology 148: 187-197
Baudry A, Heim M A, Dubreucq B, Caboche M, Weisshaar B,
Lepiniec L (2004) TT2, TT8, and TTG1 synergistically specify the expression of BANYULS and proanthocyanidin biosynthesis in Arabidopsis thaliana. Plant J 39: 366-80
Baxter I R, Young J C, Armstrong G, Foster N, Bogenschutz N, Cordova T, Peer W A, Hazen S P, Murphy A S, Harper J F (2005) A plasma membrane H+-ATPase is required for the formation of proanthocyanidins in the seed coat endothelium of Arabidopsis thaliana. PNAS 102: 5635-5635
Bogs J, Downey M, Harvey J S, Ashton A R, Tanner G J Robinson S P (2005). Proanthocyanidin synthesis and expression of genes encoding leucoanthocyanidin reductase and anthocyanidin reductase in developing grape berries and grapevine leaves. Plant Physiol. 139: 652-663.
Bogs J, Ebadi A, McDavid D, Robinson S P (2006) Identification of the flavonoid hydroxylases from grapevine and their regulation during fruit development. Plant Physiol 140: 279-291
Boss P K, Davies C, Robinson S P (1996) Analysis of the expression of anthocyanin pathway genes in developing Vitis vinifera L. cv Shiraz grape berries and the implications for pathway regulation. Plant Physiol 111: 1059-1066
Clough S. and Bent A F (1998) Floral dip: a simplified method for Agrobacterium-mediated transformation of Arabidopsis thaliana. Plant J. 16: 735-743
Cos P, De Bruyne T, Hermans N, Apers S, Berghe D V, Vlietinck A J (2004) Proanthocyanidins in health care: Current and new trends. Curr Med Chem 11: 1345-1359
Debeaujon I, Léon-Kloosterziel K M, Koornneef M. (2000). Influence of the testa on seed dormancy, germination, and longevity in Arabidopsis. Plant Physiol 122, 403-414.
Debeaujon I, Peeters A J M, Léon-Kloosterziel K M, Koornneef M (2001) The TRANSPARENT TESTA12 gene of Arabidopsis encodes a multidrug secondary transporter-like protein required for flavonoid sequestration in vacuoles of the seed coat endothelium. Plant Cell 13: 853-871
Debeaujon I, Nesi N, Perez P, Devic M, Grandjean O, Caboche M, Lepiniec L (2003) Proanthocyanidin-accumulating cells in Arabidopsis testa: Regulation of differentiation and role in seed development. Plant Cell 15: 2514-2531
Deluc L, Barrieu F, Marchive C, Lauvergeat V, Decendit A, Richard T, Cards J P, Merillon J M, Hamdi S (2006) Characterization of a grapevine R2R3-MYB transcription factor that regulates the phenylpropanoid pathway. Plant Physiol 140: 499-511
Dixon R A, Xie D Y, Sharma S B (2005) Proanthocyanidins—a final frontier in flavonoid research? New Phytologist 165: 9-28
Dixon R A, Achnine L, Kota P, Liu C J, Reddy M S S, Wang L J (2002) The phenylpropanoid pathway and plant defence—a genomics perspective. Mol Plant Pathol 3: 371-390
Dixon R A, Lamb C J, Masoud S, Sewalt V J H, Paiva N L (1996) Metabolic engineering: Prospects for crop improvement through the genetic manipulation of phenylpropanoid biosynthesis and defense responses—A review. Gene 179: 61-71
Downey M O, Harvey J S, Robinson S P (2003a) Analysis of tannins in seeds and skins of Shiraz grapes throughout berry development. Aust J Grape Wine Res 9: 15-27
Downey M O, Harvey J S, and Robinson S P (2003b) Synthesis of flavonols and expression of flavonol synthase genes in developing grape berries of Shiraz and Chardonnay (Vitis vinifera L.), Aust J Grape Wine Res 9: 110-121
Downey M O Harvey J S and Robinson S P (2004). The effect of bunch shading on berry development and flavonoid accumulation in Shiraz grapes. Aust. J. Grape Wine Res. 10:55-73
Gamborg, O J and Eveleigh, D E (1960) Culture methods and glucanases in suspension cultures of wheat and barley. Can. J. Biochem. 46:417-43.
Garfunkle (1980) Agrobacterium tumefaciens mutants affected in crown gall tumorigenesis and octopine catabolism. J. Bacteriol. 144:732-43
Gleave A P (1992) A versatile binary vector system with a T-DNA organisational structure conducive to efficient integration of cloned DNA into the plant genome. Plant Mol. Biol. 20:1203-1207
Glories Y (1988) Anthocyanins and tannins from wine: organoleptic properties. In V Cody, et al., eds, Plant Flavonoids in Biology and Medicine II: Biochemical, Cellular, and Medicinal Properties. Alan R. Liss, Inc., New York, pp 123-134
Gollop R, Farhi S, Peri A (2001) Regulation of the leucoanthocyanidin dioxygenase gene expression in Vitis vinifera. Plant Sci 161: 579-588
Higo K Y, Ugawa Iwamoto M, and Korenaga T (1999) Plant cis-acting regulatory DNA elements (PLACE) database. Nucleic Acids Res 27: 297-300.
Holton T A, Cornish E C (1995) Genetics and biochemistry of anthocyanin biosynthesis. Plant Cell 7: 1071-1083
Horsche R A, Rogers S G and Fraley R T. (1985). Transgenic Plants. Cold Spring Harb. Symp. Qant. Biol. 50:433-437
Horstmann V, Huether C M, Jost W, Reski R, Decker E L (2004) Quantitative promoter analysis in Physcornitrella patens: a set of plant vectors activating gene expression within three orders of magnitude. Bmc Biotechnol 4: 13 Jin H and Martin C (1999) Multifunctionality and diversity within the plant MYB-gene family. Plant Mol Biol 41: 577-585.
Johnson C S, Kolevski B, Smyth D R (2002) TRANSPARENT TESTA GLABRA2, a trichome and seed coat development gene of Arabidopsis, encodes a WRKY transcription factor. Plant Cell 14: 1359-1375
Kennedy J A, Matthews M A, Waterhouse A L (2000) Changes in grape seed polyphenols during ripening. Phytochemistry 55: 77-85
Kennedy J A, Hayasaka Y, Vidal S, Waters E J, Jones G P (2001) Composition of grape skin proanthocyanidins at different stages of berry development. J Agricul Food Chem 49: 5348-5355
Kitamura S, Shikazono N, Tanaka A (2004) TRANSPARENT TESTA 19 is involved in the accumulation of both anthocyanins and proanthocyanidins in Arabidopsis. Plant J 37: 104-114
Kobayashi S, Ishimaru M, Ding C K, Yakushiji H, Goto N (2001) Comparison of UDP-glucose:flavonoid 3-O-glucosyltransferase (UFGT) gene sequences between white grapes (Vitis vinifera) and their sports with red skin. Plant Sci 160; 543-550
Kobayashi S, Ishimaru M, Hiraoka K, Honda C (2002) Myb-related genes of the Kyoho grape (Vitis labruscana) regulate anthocyanin biosynthesis. Planta 215: 924-933
Kumar S, Tamura K, Nei M (2004) MEGA3: Integrated software for Molecular Evolutionary Genetics Analysis and sequence alignment. Briefings in Bioinformatics 5: 150-163
Larkin P J, at al., (1996) Transgenic White Clover. Studies with the auxin responsive promoter, GH3, in root gravitropism and lateral root development. Transgenic Res. 5:325-335.
Lazo G R, et al., (1991). A transformation-competent Arabidopsis genomic library in Agrobacterium. Bio/Technology 9:963-967
McMahon L R, McAllister T A, Berg B P, Majak W, Acharya S N, Popp J D, Coulman B E, Wang Y, Cheng K J (2000) A review of the effects of forage condensed tannins on ruminal fermentation and bloat in grazing cattle. Can J Plant Sci 80: 469-485
Mehrtens F, Kranz H, Bednarek P, Weisshaar B (2005) The Arabidopsis transcription factor MYB12 is a flavonol-specific regulator of phenylpropanoid biosynthesis. Plant Physiol 138: 1083-1096
Middleton, EJR, Kandaswami C, Theoharidis T C (2000) The effects of plant flavonoids on mammalian cells: implications for inflammation, heart disease and cancer Pharmacol Rev 52: 673-751
Mol J, Grotewold E, Koes R (1998) How genes paint flowers and seeds. Trends Plant Sci 3: 212-217
Muller P Y, Janovjak H, Miserez A R, Dobbie Z (2002) Processing of gene expression data generated by quantitative real-time RT-PCR. Biotechniques 32: 1372-1379
Nagel C W, Glories Y (1991) Use of a modified dimethylaminocinnamaldehyde reagent for analysis of flavonols. Am J Enol Viti 42: 364-366
Nesi N, Debeaujon I, Jond C, Pelletier O, Caboche M, Lepiniec L (2000) The TT8 gene encodes a basic helix-loop-helix domain protein required for expression of DFR and BAN genes in Arabidopsis siliques. Plant Cell 12: 1863-1878
Nesi N, Jond C, Debeaujon I, Caboche M, Lepiniec L (2001) The Arabidopsis TT2 gene encodes an R2R3 MYB domain protein that acts as a key determinant for proanthocyanidin accumulation in developing seed. Plant Cell 13: 2099-2114
Nesi N, Debeaujon I, Jond C, Stewart A J, Jenkins G I, Caboche M, Lepiniec L (2002) The TRANSPARENT TESTA16 locus encodes the ARABIDOPSIS BSISTER MADS domain protein and is required for proper development and pigmentation of the seed coat. Plant Cell 14: 2463-2479
Peters D J and Constabel C P (2002) Molecular analysis of herbivore-induced condensed tannin synthesis: cloning and expression of dihydroflavonol reductase from trembling aspen (Populus tremuloides). Plant J 32: 701-712
Phillips G C and Collins G B (1984) Red clover and other forage legumes. In Sharp, W R, Evans, D A, Ammirato P V and Yamada Y, eds., Handbook of Plant Cell Culture Vol. 2 Crop Species, pp. 169-210. New York; Macmillan Publishing
Pourcel L, Routaboul J M, Kerhoas L, Caboche M, Lepiniec L, Debeaujon I (2005) TRANSPARENT TESTA10 encodes a laccase-like enzyme involved in oxidative polymerization of flavonoids in Arabidopsis seed coat. Plant Cell 17: 2966-2980
Quattrocchio F, Wing J F, van der Woude K, Mol J N M, Koes R (1998) Analysis of bHLH and MYB domain proteins: Species specific regulatory differences are caused by divergent evolution of target anthocyanin genes. Plant J 13: 475-488
Quattrocchio F, Wing J, van der Woude K, Souer E, de Vetten N, Mol J, Koes R (1999) Molecular analysis of the anthocyanin2 gene of Petunia and its role in the evolution of flower color. Plant Cell 11: 1433-1444
Quackenbush J, Liang F, Holt I, Pertea G, Upton J (2000) The TIGR Gene Indices: reconstruction and representation of expressed gene sequences. Nucleic Acids Res 28: 141-145
Ramsay N A, Walker A R, Mooney M, Gray J C (2003) Two basic-helix-loop-helix genes (MYC-146 and GL3) from Arabidopsis can activate anthocyanin biosynthesis in a white-flowered Matthiola incana mutant. Plant Mol Biol 52: 679-688
Robinson S P, Davies C (2000) Molecular biology of grape berry ripening. Aust J Grape Wine Res 6: 175-188
Sagasser M, Lu G H, Hahlbrock K, Weisshaar B (2002) A-thaliana TRANSPARENT TESTA 1 is involved in seed coat development and defines the WIP subfamily of plant zinc finger proteins. Genes Dev 16: 138-149
Sharma S B, Dixon R A (2005) Metabolic engineering of proanthocyanidins by ectopic expression of transcription factors in Arabidopsis thaliana. Plant J 44: 62-75
Shirley B W, Hanley S, Goodman H M (1992) Effects of ionizing radiation on a plant genome: analysis of two Arabidopsis transparent testa mutations. Plant Cell 4: 333-347
Shirley B W, Kubasek W L, Storz G, Bruggemann E, Koornneef M, Ausubel F M, Goodman H M (1995) Analysis of Arabidopsis mutants deficient in flavonoid biosynthesis. Plant J 8: 659-671
Solano R, Nieto C, Pazares J (1995) Myb.Ph3 transcription factor from Petunia hybrida induces similar DNA-bending/distortions on its 2 types of binding-site. Plant J 8: 673-682
Sparvoli F, Martin C, Scienza A, Gavazzi G, Tonelli C (1994) Cloning and molecular analysis of structural genes involved in flavonoid and stilbene biosynthesis in grape (Vitis vinifera L.). Plant Mol Biol 24: 743-755
Stracke R, Werber M, Weisshaar B (2001) The R2R3-MYB gene family in Arabidopsis thaliana. Curr Opin Plant Biol 4: 447-456
Torregrosa L, Verries C, Tesniere C (2002) Grapevine (Vitis vinifera L.) promoter analysis by biolistic-mediated transient transformation of cell suspensions. Vitis 41: 27-32
Voisey C R et al. (1994) Agrobacterium-mediated transformation of white clover using direct shoot organogenesis. Plant Cell Rep. 13:309-314
Walker A R, Davison P A, Bolognesi-Winfield A C, James C M, Srinivasan N, Blundell T L, Esch J J, Marks M D, Gray J C (1999) The TRANSPARENT TESTA GLABRA1 locus, which regulates trichome differentiation and anthocyanin biosynthesis in Arabidopsis, encodes a WD40 repeat protein. Plant Cell 11: 1337-1349
Winkel-Shirley B (2001) Flavonoid biosynthesis. A colorful model for genetics, biochemistry, cell biology, and biotechnology. Plant Physiol 126: 485-493
Xie D Y, Sharma S B, Paiva N L, Ferreira D, Dixon R A (2003) Role of anthocyanidin reductase, encoded by BANYULS in plant flavonoid biosynthesis. Science 299: 396-399
Xie D Y, Sharma S B, Wright E, Wang Z Y, Dixon R A (2006) Metabolic engineering of proanthocyanidins through co-expression of anthocyanidin reductase and the PAP1 MYB transcription factor. Plant J 45: 895-907
Zimmermann I M, Heim M A, Weisshaar B, Uhrig J F (2004) Comprehensive identification of Arabidopsis thaliana MYB transcription factors interacting with R/B-like BHLH proteins. Plant J 40: 22-34.

1-28. (canceled)

29. A method for identifying a homologue of the VvMYBPA1 protein that increases synthesis of proanthocyanidin in a plant cell comprising:
i) introducing into a plant cell a non-endogenous nucleic acid molecule which encodes the homologue of the VvMYBPA1 protein, wherein the homologue of the VvMYBPA1 protein includes amino acids in a sequence having at least 40% sequence identity to amino acids 116-286 of the VvMYBPA1 protein sequence set forth in SEQ ID NO: 2;
ii) growing the plant cell or its progeny cells under conditions to express the non-endogenous nucleic acid molecule; and
iii) determining whether the cell or its progeny cells expressing the non-endogenous nucleic acid molecule have increased synthesis of a proanthocyanidin relative to a plant cell of the same species not expressing the non-endogenous nucleic acid molecule,
wherein an increased level of proanthocyanidin synthesis in the cell or its progeny cells expressing the non-endogenous nucleic acid molecule relative to a plant cell of the same species not expressing the non-endogenous nucleic acid molecule identifies the VvMYBPA1 homologue as a VvMYBPA1 homologue that increases the synthesis of proanthocyanidin in a plant cell.

30. A method for identifying a plant cell that produces an increased level of proanthocyanidin as a result of expression of a homologue of the VvMYBPA1 protein in the plant cell comprising:
i) introducing into a plant cell a non-endogenous nucleic acid molecule which encodes the homologue of the VvMYBPA1 protein, wherein the homologue of the VvMYBPA1 protein includes amino acids in a sequence having at least 40% sequence identity to amino acids 116-286 of the VvMYBPA1 protein sequence set forth in SEQ ID NO: 2 and wherein the homologue of the VvMYBPA1 protein is produced in a plant species which produces proanthocyanidin;
ii) growing the plant cell or its progeny cells under conditions to express the non-endogenous nucleic acid molecule; and
iii) determining whether the cell or its progeny cells expressing the non-endogenous nucleic acid molecule have increased synthesis of a proanthocyanidin relative to a plant cell of the same species not expressing the non-endogenous nucleic acid molecule,
wherein an increased level of proanthocyanidin synthesis in the cell or its progeny cells expressing the non-endogenous nucleic acid molecule relative to a plant cell of the same species not expressing the non-endogenous nucleic acid molecule identifies the plant cell as a plant cell that produces an increased level of proanthocyanidin as a result of expression of the homologue of the VvMYBPA1 protein in the plant cell.
